(19) United States
(12) Patent Application Publication
(10) Pub. No.: US 2026/0284149 A1
(43) Pub. Date: Sep. 24, 2026

(54) TARGETING THE IGF-1 PATHWAY TO EXTEND ORGAN LIFESPAN

(71) Applicant: The Regents of the University of Michigan, Ann Arbor, MI (US)

(72) Inventors: Abhijit S. Naik, Ann Arbor, MI (US); Roger C. Wiggins, Ann Arbor, MI (US)

(21) Appl. No.: 19/475,464

(22) PCT Filed: Apr. 18, 2024
(86) PCT No.: PCT/US2024/025164

§ 371 Date: Oct. 15, 2025

Related U.S. Application Data
(60) Provisional application No. 63/460,140, filed on Apr. 18, 2023.

(51) Int. Cl. A61K 38/27; A61K 31/05; A61K 31/36; A61K 31/4706; A61K 31/4985; A61K 31/506; A61K 31/517; A61K 31/519; A61K 31/5377; A61K 31/675; A61K 38/28; A61K 39/395; A61P 37/06

(52) U.S. Cl. CPC A61K 38/27; A61K 31/05; A61K 31/36; A61K 31/4706; A61K 31/4985; A61K 31/506; A61K 31/517; A61K 31/519; A61K 31/5377; A61K 31/675; A61K 38/28; A61K 39/3955; A61P 37/06

(57) ABSTRACT

Provided herein are compositions and methods for extending organ lifespan, including extending the lifespan of a transplanted organ. The compositions and methods involve providing to an organ before transplant or providing to a subject receiving a transplant an inhibitor of IGF-1R signaling and/or an inhibitor of GHR signaling.

### PRIORITY STATEMENT

This application claims priority to U.S. Provisional Application No. 63/460,140, filed Apr. 18, 2023, the entire contents of which are incorporated herein by reference for all purposes.

### STATEMENT REGARDING FEDERAL FUNDING

This invention was made with government support under DK046073, DK081943, DK102643, and DK125529 awarded by the National Institutes of Health. The government has certain rights in the invention.

### FIELD

Provided herein are compositions and methods for extending organ lifespan, including extending the lifespan of a transplanted organ. The compositions and methods involve providing to an organ before transplant or providing to a subject receiving a transplant an inhibitor of IGF-1R signaling and/or an inhibitor of GHR signaling.

### BACKGROUND

The average kidney transplant (allograft) lasts only 15 years when it would be expected to last considerably longer. Depletion of podocytes, which are neuron-like cells in the glomerular filter of the kidney, is a determinant of many types of kidney failure. Kidney transplant leads to adaptive enlargement (hypertrophy) of the transplanted kidney. This adaptive enlargement causes podocyte hypertrophy and stress and leading to accelerated podocyte detachment and depletion from glomeruli, leading to progressive glomerular scarring (glomerulosclerosis), loss of kidney function, and kidney failure. Similar hyperemia and hypertrophy occur in other transplants, including heart and lung. Accordingly, what is needed are methods for increasing the lifespan of kidney transplants.

### SUMMARY

In some aspects, provided herein are compositions and methods for extending lifespan of an organ, including a damaged or diseased organ and an organ following transplant. In some aspects, provided herein are methods for determining the lifespan of an organ for transplant, and for improving the lifespan for subject in need thereof.

In some aspects, provided herein are methods of extending lifespan of a transplant. In some embodiments, provided herein is a method of extending lifespan of a transplant comprising providing an inhibitor of insulin-like growth factor 1 receptor (IGF-1R) signaling and/or an inhibitor of growth hormone receptor (GHR) signaling to a subject before, during, and/or after a transplant in the subject. In some embodiments, the transplant is a kidney transplant, a lung transplant, a heart transplant, or another organ transplant. For example, in some embodiments the transplant is a kidney transplant.

In some embodiments, the inhibitor of IGF-1R signaling and/or the inhibitor of GHR signaling is provided to the subject within 30 days of the transplant. In some embodiments, the inhibitor of IGF-1R signaling and/or the inhibitor of GHR signaling is provided to the subject within 3 weeks of the transplant. In some embodiments, the inhibitor of IGF-1R signaling and/or the inhibitor of GHR signaling is provided to the subject within 2 weeks of the transplant. In some embodiments, the inhibitor of IGF-1R signaling and/or the inhibitor of GHR signaling is provided to the subject within 10 days of the transplant. In some embodiments, the inhibitor of IGF-1R signaling and/or the inhibitor of GHR signaling is provided to the subject within 7 days of the transplant. In some embodiments, the inhibitor of IGF-1R signaling and/or the inhibitor of GHR signaling is provided to the subject within 5 days of the transplant. In some embodiments, the inhibitor of IGF-1R signaling and/or the inhibitor of GHR signaling is provided to the subject within 3 days of the transplant. In some embodiments, the IGF-1R inhibitor and/or the inhibitor of GHR signaling is provided to the subject prior to transplantation, at the time of transplantation, or after transplantation. For example, in some embodiments the inhibitor of IGF-1R signaling and/or the inhibitor of GHR signaling is provided to the subject within 3 weeks after transplant, within 1 week after transplant, within 5 day safter transplant, within 3 days after transplant, within 2 days after transplant, within 1 day of transplant, within 12 hours after transplant, within 6 hours after transplant, within 3 hours after transplant, immediately after transplant (e.g. within 1 hour after transplant), or during transplant.

In some embodiments, the method of extending lifespan of a transplant comprises providing an inhibitor of IGF-1R signaling to the subject. In some embodiments, the inhibitor of IGF-1R signaling comprises a small molecule inhibitor. Suitable small molecule inhibitors of IGF-1R signaling include, for example, Linsitinib, NVP-AEW541, GSK1904529A, NVP-ADW742, BMS-536924, Ceritinib, AG-1024, GSK1838705A, BMS-754807, PQ 401, Ginsenoside Rg5, Ceritinib dihydrochloride, XL228, Brigatinib, Picropodophyllin, AZD3463, Insulin Degludec, S961, Nordihydroguaiaretic acid (NGDA), and combinations thereof. In some embodiments, the inhibitor of IGF-1R signaling comprises an antibody. Suitable antibodies include, for example, Teprotumumab, Dalotuzumab, Figitumumab, Ganitumab, Robatumumab, Cixutumumab, and combinations thereof.

In some embodiments, the method of extending lifespan of a transplant comprises providing an inhibitor of GHR signaling to the subject. In some embodiments, the inhibitor of GHR signaling is a protein or an antibody. In some embodiments, the inhibitor of GHR signaling is Pegvisomant. The subject may be an adult subject, a peripubertal subject, or a prepubertal subject.

In some embodiments, the transplant is a kidney transplant, and levels of IGF-1 in a sample obtained from the subject prior to kidney transplant are above a threshold value for IGF-1 and/or an effective kidney dose (eKD) of the kidney transplant is below a threshold value for eKD.

In some aspects, provided herein are methods of determining potential lifespan of a kidney transplant in a subject. In some embodiments, methods of determining potential lifespan of a kidney transplant in a subject comprise determining the effective kidney dose (eKD) of the kidney transplant, determining levels of IGF-1 in a sample obtained from the subject, and determining the potential lifespan of the kidney transplant in the subject based at least in part upon the eKD and the levels of IGF-1 in the sample obtained from the subject. In some embodiments, the method of determining potential lifespan of a kidney transplant in a subject further comprises determining one or more factors selected from age of the organ donor, status of the organ donor, age of the subject, and race of subject, wherein the one or more factors are additionally used in the determination of potential lifespan of the kidney transplant. In some embodiments, the potential lifespan of the kidney transplant in the subject is determined based at least in part upon one of more of the eKD, the levels of IGF-1 in the sample obtained from the subject, age of the organ donor, status of the organ donor, age of the subject, race of subject. In some embodiments, the potential lifespan of the kidney transplant in the subject is determined based at least in part upon two of more of the eKD, the levels of IGF-1 in the sample obtained from the subject, age of the organ donor, status of the organ donor, age of the subject, race of subject. In some embodiments, the potential lifespan of the kidney transplant in the subject is determined based at least in part upon three of more of the eKD, the levels of IGF-1 in the sample obtained from the subject, age of the organ donor, status of the organ donor, age of the subject, race of subject. In some embodiments, additional considerations are used in the determination of the potential lifespan of the kidney transplant in the subject. In some embodiments, the potential lifespan of the kidney transplant is determined to be acceptable. In some embodiments, the potential lifespan of the kidney transplant is determined to be poor. In some embodiments, the method further comprises providing an inhibitor of IGF-1R signaling and/or an inhibitor of GHR signaling to the subject when the lifespan of the kidney transplant is determined to be poor.

In some embodiments, the method of determining potential lifespan of a kidney transplant in a subject comprises determining a poor lifespan of the kidney transplant in the subject when levels of IGF-1 in the sample are equal to or above a threshold value and the eKD is below a threshold value. In some embodiments, the method of determining potential lifespan of a kidney transplant in a subject comprises determining an acceptable lifespan of the kidney transplant when the eKD is equal to or above a threshold value. In some embodiments, the method of determining potential lifespan of a kidney transplant in a subject comprises determining an acceptable lifespan of the kidney transplant when the levels of IGF-1 in the sample are below a threshold value.

In some embodiments, the method further comprises providing an inhibitor of insulin-like growth factor 1 receptor (IGF-1R) signaling and/or an inhibitor of growth hormone receptor (GHR) signaling to the subject when a poor lifespan of the kidney transplant is determined. In some embodiments, the inhibitor of IGF-1R signaling and/or the inhibitor of GHR signaling is provided to the subject within 30 days of the kidney transplant. In some embodiments, the inhibitor of IGF-1R signaling and/or the inhibitor of GHR signaling is provided to the subject within 3 weeks of the kidney transplant. In some embodiments, the inhibitor of IGF-1R signaling and/or the inhibitor of GHR signaling is provided to the subject within 2 weeks of the kidney transplant. In some embodiments, the inhibitor of IGF-1R signaling and/or the inhibitor of GHR signaling is provided to the subject within 10 days of the kidney transplant. In some embodiments, the inhibitor of IGF-1R signaling and/or the inhibitor of GHR signaling is provided to the subject within 7 days of the kidney transplant. In some embodiments, the inhibitor of IGF-1R signaling and/or the inhibitor of GHR signaling is provided to the subject within 5 days of the kidney transplant. In some embodiments, the inhibitor of IGF-1R signaling and/or the inhibitor of GHR signaling is provided to the subject within 3 days of the kidney transplant. In some embodiments, the method comprises providing an inhibitor of IGF-1R signaling to the subject. In some embodiments, the inhibitor of IGF-1R signaling comprises a small molecule inhibitor. Suitable small molecule inhibitors of IGF-1R signaling include, for example, Linsitinib, NVP-AEW541, GSK1904529A, NVP-ADW742, BMS-536924, Ceritinib, AG-1024, GSK1838705A, BMS-754807, PQ 401, Ginsenoside Rg5, Ceritinib dihydrochloride, XL228, Brigatinib, Picropodophyllin, AZD3463, Insulin Degludec, S961, Nordihydroguaiaretic acid (NGDA), and combinations thereof. In some embodiments, the inhibitor of IGF-1R signaling comprises an antibody. Exemplary antibodies include, for example, Teprotumumab, Dalotuzumab, Figitumumab, Ganitumab, Robatumumab, Cixutumumab, and combinations thereof. In some embodiments, the method comprises providing an inhibitor of GHR signaling to the subject. In some embodiments, the inhibitor of GHR signaling is a protein or an antibody. For example, in some embodiments the inhibitor of GHR signaling is

### Pegvisomant.

In some aspects, provided herein are methods of extending lifespan of a transplant comprising contacting a donor organ obtained from a deceased subject with an inhibitor of insulin-like growth factor 1 receptor (IGF-1R) signaling and/or an inhibitor of growth hormone receptor (GHR) signaling prior to transplant of the donor organ into a subject. In some embodiments, the donor organ is a heart, a lung, or a kidney. In some embodiments, the donor organ is a kidney. In some embodiments, contacting the donor organ with the inhibitor comprises perfusing the donor organ with a composition comprising the inhibitor of IGF-1R signaling and/or the inhibitor of GHR signaling. In some embodiments, the method comprises contacting the donor organ with an inhibitor of IGF-1R signaling. In some embodiments, the inhibitor of IGF-1R signaling comprises a small molecule inhibitor. Suitable small molecule inhibitors of IGF-1R signaling include, for example, Linsitinib, NVP-AEW541, GSK1904529A, NVP-ADW742, BMS-536924, Ceritinib, AG-1024, GSK1838705A, BMS-754807, PQ 401, Ginsenoside Rg5, Ceritinib dihydrochloride, XL228, Brigatinib, Picropodophyllin, AZD3463, Insulin Degludec, S961, Nordihydroguaiaretic acid (NGDA), and combinations thereof. In some embodiments, the inhibitor of IGF-1R signaling comprises an antibody. Suitable antibodies include, for example, Teprotumumab, Dalotuzumab, Figitumumab, Ganitumab, Robatumumab, Cixutumumab, and combinations thereof. In some embodiments, the method comprises contacting the donor organ with an inhibitor of GHR signaling. In some embodiments, the inhibitor of GHR signaling is a protein or an antibody. For example, in some embodiments the inhibitor of GHR signaling is Pegvisomant.

In some aspects, provided herein are methods of extending kidney lifespan in a subject having kidney dysfunction. In some embodiments, methods of extending kidney lifespan in a subject having kidney dysfunction comprise providing an inhibitor of insulin-like growth factor 1 receptor (IGF-1R) signaling and/or an inhibitor of growth hormone receptor (GHR) signaling to a subject having kidney dysfunction. In some embodiments, the kidney dysfunction is selected from hyperfiltration, glomerular injury, reduced nephron number, and glomerular disease. In some embodiments, the kidney dysfunction is hyperfiltration. For example, in some embodiments the kidney dysfunction is hyperfiltration induced by hypertension, partial nephrectomy, kidney transplantation, chronic kidney disease, diabetes, and/or obesity. In some embodiments, the kidney dysfunction is a glomerular disease. For example, in some embodiments the glomerular disease is focal segmental glomerulosclerosis (FSGS), acute glomerulonephritis, crescentic glomerulonephritis, IgA nephropathy, lupus nephritis, cryoglobulinemia, Membranoproliferative glomerulonephritis (MPGN) transplant glomerulopathy, membranous glomerulopathy, Genetic glomerular disease, and/or minimal change disease.

In some embodiments, the method of extending kidney lifespan in a subject having kidney dysfunction comprises providing an inhibitor of IGF-1R signaling to the subject. In some embodiments, the inhibitor of IGF-1R signaling comprises a small molecule inhibitor. Suitable small molecule inhibitors of IGF-1R signaling include, for example, Linsitinib, NVP-AEW541, GSK1904529A, NVP-ADW742, BMS-536924, Ceritinib, AG-1024, GSK1838705A, BMS-754807, PQ 401, Ginsenoside Rg5, Ceritinib dihydrochloride, XL228, Brigatinib, Picropodophyllin, AZD3463, Insulin Degludec, S961, Nordihydroguaiaretic acid (NGDA), and combinations thereof. In some embodiments, the inhibitor of IGF-1R signaling comprises an antibody. Suitable antibodies include, for example, Teprotumumab, Dalotuzumab, Figitumumab, Ganitumab, Robatumumab, Cixutumumab, and combinations thereof. In some embodiments, the method of extending kidney lifespan in a subject having kidney dysfunction comprises providing an inhibitor of GHR signaling to the subject. In some embodiments, the inhibitor of GHR signaling is a protein or an antibody. For example, in some embodiments the inhibitor of GHR signaling is Pegvisomant.

In some aspects, provided herein are uses of an inhibitor of IGF-1R signaling. In some embodiments, provided herein is a use of an inhibitor of insulin-like growth factor 1 receptor (IGF-1R) signaling in a method of extending lifespan of a transplant. In some embodiments, the transplant is from a deceased donor or a living donor.

In some aspects, provided herein are uses of an inhibitor of growth hormone receptor (GHR) signaling. In some embodiments, provide herein is a use of an inhibitor of GHR signaling in a method of extending lifespan of a transplant. In some embodiments, the transplant is from a deceased donor or a living donor.

### BRIEF DESCRIPTION OF THE DRAWINGS

FIG. 1 shows an outline of experiments and data analysis conducted herein to investigate whether kidney allograft IGF-1 exposure that occurs at transplantation is related to long-term allograft longevity. Population-level datasets (upper panel) were evaluated, along with direct testing in patient-level data (lower panel). The effective kidney dose delivered at transplantation (eKD), which determines the degree of allograft hyperfiltration, interacts with ambient IGF-1 level in the blood to determine the level of allo-immune activation and graft longevity.

FIGS. 2A-2D are graphs showing the relationship between death-censored allograft survival and pre-transplant IGF-1 level by recipient age in 1-year increments. FIG. 2A is a graph showing the relationship between median death-censored allograft survival (longevity) and recipient age at kidney transplantation. FIG. 2B is a scatterplot of the population level data from Bidlingmaier et al. showing sex-specific curves in mean serum IGF-1 levels by age, with both peaking at 16 years old. Females (circles). Males (squares). FIG. 2C is a graph showing the average serum IGF-1 level by age for females and males. FIG. 3D is a superimposition of FIG. 2A and FIG. 2C to visually illustrate the inverse relationship between average serum IGF-1 levels by recipient age and median allograft longevity at the time of transplantation.

FIGS. 3A-3E shows patient-level data. FIG. 3A shows the relationship of pre-transplant IGF-1 level with recipient age at kidney transplantation. A wide range was found across all ages, but the averaged curve is similar to that present in the reference population (shown in FIG. 2C). FIG. 3B shows the relationship of pre-transplant IGF-2 level with recipient age. A wide range of IGF-2 levels was present across all ages. FIG. 3C shows a moderate correlation between pre-transplant IGF-1 and IGF-2 levels was present (r=0.48, p<0.0001). Note that IGF-2 was below the level of detection in 27 samples (12.5%). FIG. 3D shows the relationship between donor: recipient BSA ratio and the age at kidney transplantation. This relationship is similar to that obtained for the population dataset (not shown). The vertical line denotes the 16-year age. FIG. 3E shows the relationship between pre-transplant donor BSA and recipient BSA. The lack of a significant correlation is consistent with no intentional donor-to-recipient body size matching at the time of transplantation in this cohort.

FIG. 4A-4F shows the covariate-adjusted predicted effect of IGF-1 levels at transplantation into a 40-year-old recipient on allograft failure at different Effective Kidney Doses. In each graph, the predicted curves are shown for a range of effective kidney doses transplanted (eKD), including 1^{st} % ile (≡0.5 kidneys), 10^{th} % ile (≡0.74 kidneys), 50^{th} % ile (1 kidney), 90^{th} % ile (≡1.36 kidneys) and the 99^{th} % ile (≡3.68 kidneys). IGF-1 levels at transplantation are shown as higher range (90^{th} % ile=400 ng/ml), average range (50^{th} % ile=180 ng/ml) or lower range (10^{th} % ile=92 ng/ml). FIGS. 4A, 4B, and 4C demonstrate the relationship between pretransplant recipient IGF-1 levels and allograft outcomes among kidneys from deceased donors. FIGS. 4D, 4E, and 4F demonstrate the relationship between pretransplant recipient IGF-1 levels and allograft outcomes among kidneys from living donors. These data show that the relationship between IGF-1 levels before transplantation and death-censored allograft outcomes depends on the eKD delivered at transplantation. At higher IGF-1 levels, low eKD is accompanied by inferior allograft survival compared to that of higher eKD levels. While at lower IGF-1 levels, the lower eKD group had an allograft survival comparable to high eKD groups. Thus, modulation of IGF-1 signaling or kidney dose finds use to improve long-term kidney allograft survival by mitigating IGF-1-mediated effects. Note that at low IGF-1 levels, transplantation of even a low eKD was associated with excellent survival over 10 years. Variables in the final model included recipient age, race, gender, donor age, race, gender, and type of kidney transplant (Deceased donor versus Living donor), IGF-1 levels pretransplant, eKD (estimated kidney dose), and the interaction of the last two terms. Parallel data for recipients ages 16 years (the age of peak IGF-1 levels during puberty), 20 years (10^{th} % ile), 47 (50^{th} % ile), and 67 (90^{th} % ile) for the cohort are shown in FIG. 9, FIG. 10, FIG. 11, and FIG. 12. These data demonstrate a strong additional age effect on allograft survival. The reference donor for this analysis was a 40-year-old white donor.

FIGS. 5A-5F show covariate adjusted predicted effect of IGF-1 levels at transplantation on development of post-transplant proteinuria (>=1.0 g/g) at different Effective Kidney Doses. In each graph, the predicted curves are shown for a range of effective kidney doses transplanted (eKD), including 1^{st} % ile (≡0.5 kidneys), 10^{th} % ile (≡0.74 kidneys), 50^{th} % ile (≡1 kidney), 90^{th} % ile (≡1.36 kidneys) and the 99^{th} % ile (≡3.68 kidneys). IGF-1 levels at transplantation are shown as higher range (90^{th} % ile=400 ng/ml), average range (50^{th} % ile=180 ng/ml) or lower range (10^{th} % ile=92 ng/ml). FIGS. 5A-5C demonstrate the relationship between pretransplant IGF-1 levels and proteinuria among kidneys from deceased donors. FIGS. 5D-5F demonstrate the relationship between pretransplant IGF-1 levels and proteinuria among kidneys from living donors. These data show that the relationship between IGF-1 levels before transplantation and proteinuria depends on the eKD delivered at transplantation. At higher IGF-1 levels, low eKD is accompanied by higher rates of proteinuria compared to higher eKD levels. While at lower IGF-1 levels, the lower eKD group had proteinuria comparable to high eKD groups. Thus, modulation of IGF-1 signaling or kidney dose finds use to improve long-term kidney allograft survival by mitigating post-transplant proteinuria. Note that at low IGF-1 levels, transplantation of even a low eKD was associated with the maintenance of low-level proteinuria over 10 years. Variables in the final model included recipient age, race, gender, donor age, race, gender, and type of kidney transplant (Deceased donor versus Living donor), previous transplantation, calculated panel reactive antibody % 1 and 2, IGF-1 levels pretransplant, eKD (estimated kidney dose) and the interaction of the last two terms. Parallel data for recipients ages 16 years (the age of peak IGF-1 levels during puberty), 20 years (10^{th} % ile), 47 (50^{th} % ile), and 67 (90^{th} % ile) for the cohort are shown in FIGS. 13, 14, 15, and 16. These data demonstrate a strong additional age effect on proteinuria. The reference donor for this analysis was a 40-year-old white donor.

FIGS. 6A-6F show covariate adjusted predicted effect of IGF-1 levels at transplantation on the development of the composite alloimmune responses at different Effective Kidney Doses. FIG. 6A-6C: In each graph, the predicted curves are shown for a range of effective kidney doses transplanted (eKD), including 1^{st} % ile (≡0.5 kidneys), 10^{th} % ile (≡0.74 kidneys), 50^{th} % ile (≡1 kidney), 90^{th} % ile (≡1.36 kidneys) and the 99^{th} % ile (≡3.68 kidneys). IGF-1 levels at transplantation are shown as a higher range (90^{th} % ile=400 ng/ml), average range (50^{th} % ile=180 ng/ml), or lower range (10^{th} % ile=92 ng/ml) in deceased donor transplants. FIG. 6D-6F demonstrate the relationship between pretransplant IGF-1 levels at the time of kidney transplantation and the composite alloimmune response among kidneys from living donors. These data show that the relationship between IGF-1 levels before transplantation and composite alloimmune response depends on the eKD delivered at transplantation. At higher IGF-1 levels, low eKD is accompanied by an increased composite alloimmune response compared to those with higher eKD levels. While at lower IGF-1 levels, the lower eKD group had alloimmune responses comparable to high eKD groups. Thus, modulation of IGF-1 signaling or increasing kidney dose finds use to improve long-term kidney allograft survival by mitigating injury from post-transplant alloimmune responses. Variables in the final model included recipient age, race, gender, donor age, race, gender, and type of kidney transplant (Deceased donor versus Living donor), previous transplantation, calculated panel reactive antibody % 1 and 2, IGF-1 levels pretransplant, eKD (estimated kidney dose) and the interaction of the last two terms. The reference donor for this analysis was a 40-year-old white donor.

FIGS. 7A-7F show covariate adjusted predicted effect of IGF-1 levels at transplantation on the time to development of T-cell mediated rejection (TCMR) at different Effective Kidney Doses. FIG. 7A-7C: In each graph, the predicted curves are shown for a range of effective kidney doses transplanted (eKD), including 1^{st} % ile (≡0.5 kidneys), 10^{th} % ile (≡0.74 kidneys), 50^{th} % ile (≡1 kidney), 90^{th} % ile (≡1.36 kidneys) and the 99^{th} % ile (≡3.68 kidneys). IGF-1 levels at transplantation are shown as higher range (90^{th} % ile=400 ng/ml), average range (50^{th} % ile=180 ng/ml) or lower range (10^{th} % ile=92 ng/ml). FIGS. 7D-7F demonstrate the relationship between pretransplant IGF-1 levels at the time of kidney transplantation and TCMR among kidneys from living donors. These data show that the relationship between IGF-1 levels before transplantation and TCMR depends on the eKD delivered at transplantation. At higher IGF-1 levels, low eKD is accompanied by higher TCMR rates compared to those with higher eKD levels. While at lower IGF-1 levels, the lower eKD group had TCMR rates comparable to high eKD groups. Thus, modulation of IGF-1 signaling or increasing kidney dose finds use to improve long-term kidney allograft survival by mitigating injury from post-transplant TCMR. Note that at low IGF-1 levels, transplantation of even a low eKD was associated with low TCMR rates over 10 years. Variables in the final model included recipient age, race, gender, donor age, race, gender, and type of kidney transplant (Deceased donor versus Living donor), previous transplantation, calculated panel reactive antibody % 1 and 2, IGF-1 levels pretransplant, eKD (estimated kidney dose) and the interaction of the last two terms. Parallel data for recipients ages 16 years (the age of peak IGF-1 levels during puberty), 20 years (10^{th} % ile), 47 (50^{th} % ile), and 67 (90^{th} % ile) for the cohort are shown in FIGS. 17, 18, 19, and 20. These data demonstrate a strong additional age effect on TCMR. The reference donor for this analysis was a 40-year-old white donor.

FIG. 8 is a diagrammatic illustration of concepts developed herein by which allografts can become exposed to excess IGF-1 that drives various pathways identified to be associated with worse allograft outcomes. Pathways driven by excess kidney exposure to IGF-1 are shown in orange. Lower kidney dose and higher IGF-1 levels that interact to drive these pathways are shown in pink. The protective effect of lower IGF-1 levels and a higher kidney dose are shown in green.

FIGS. 9A-9F show parallel data to that presented in FIGS. 4A-4F for recipients ages 16 years (the age of peak IGF-1 levels during puberty) of age.

FIGS. 10A-10F show parallel data to that presented in FIGS. 4A-4F for recipients ages 20 years (10^{th} % ile).

FIGS. 11A-11F show parallel data to that presented in FIGS. 4A-4F for recipients ages 47 years (50^{th} % ile).

FIGS. 12A-12F show parallel data to that presented in FIGS. 4A-4F for recipients ages 67 years (90^{th} % ile).

FIGS. 13A-13F show parallel data to that presented in FIGS. 5A-5F for recipients ages 16 years (the age of peak IGF-1 levels during puberty).

FIGS. 14A-14F show parallel data to that presented in FIGS. 5A-5F for recipients ages 20 years (10^{th} % ile).

FIGS. 15A-15F show parallel data to that presented in FIGS. 5A-5F for recipients ages 47 years (50^{th} % ile).

FIGS. 16A-16F show parallel data to that presented in FIGS. 5A-5F for recipients ages and 67 (90^{th} % ile).

FIGS. 17A-17F show parallel data to that presented in FIGS. 7A-7F for recipients ages 16 years (the age of peak IGF-1 levels during puberty).

FIGS. 18A-18F show parallel data to that presented in FIGS. 7A-7F for recipients ages 20 years (10^{th} % ile).

FIGS. 19A-19F show parallel data to that presented in FIGS. 7A-7F for recipients ages 47 years (50^{th} % ile).

FIGS. 20A-20F show parallel data to that presented in FIGS. 7A-7F for recipients ages 67 years (90^{th} % ile)

FIG. 21 shows single cell transcriptomic analysis of IGF-pathway transcripts in kidney cells derived from normal kidney biopsies (left panels) and allograft biopsies showing no histologic abnormality (right panels). Data are from a previously reported dataset, wherein the criteria for cell identification are provided (Menon R et al. Kidney Int. 2022; 101(4):779-792) To the right of each panel, the number of cells available for analysis for each cell type is shown. The cell types are presented in alphabetical order from top to bottom (definitions below). For each transcript, the dot size denotes the proportion of cells within the cell type that expressed a detectable transcript. The dot color's intensity indicates the average amount of transcript detected across all cells. Calibrations for dot size and intensity are displayed at the lower right of each panel. Note that the calibrations for the upper panels differ, while those for the lower panels are identical. The upper panels display transcripts for Insulin-like Growth Factor-1 (IGF1), IGF-1 Receptor (IGF1R), Growth Hormone Receptor (GHR), Insulin-like Growth Factor-2 (IGF2), and Insulin Receptor (INSR). The lower panels exhibit transcripts for Insulin-like Growth Factor Binding Proteins 1-6 (IGFBP1-6). Cell designations are as follows: ATL (Ascending Thin Limb), BCells (B Cells), CNT (Connecting Tubule), DC (Dendritic Cells), DCT (Distal Convoluted Tubule), DTL (Descending Thin Limb), EC1 (Arteriolar Endothelial Cells), EC2 (Glomerular Endothelial Cells), EC3 (Peritubular Capillaries), FIB (Fibroblasts), IC1/2 (Intercalated Cells 1 and 2), MAC (Macrophages), MC (Mesangial Cells), MON (Monocytes), NKC (Natural Killer Cells), NKT (Natural Killer T Cells), PC1/2 (Principal Cells Cluster 1 and 2), POD (Podocytes), PT/DTL (Proximal Tubule/Descending Thin Limb), PT1/2/3 (Proximal Tubule Sub-clusters 1, 2, or 3), T-ACT (Activated T cells), TAL1/2 (Thick Ascending Limb 1 or 2 sub-cluster), TCells (T Cells), tDCT_CNT (Transition cells of DCT with CNT), tPC_IC (Transitional Principal and Intercalated Cells), tDAL_DCT (Transition cells between TAL and DCT), vSMC (Vascular Smooth Muscle Cells). It is noted that in normal kidneys, podocytes express higher levels of transcripts for IGF-1, IGF-1R, and GHR, consistent with their role in an intrinsic IGF-1 production and negative feedback loop, which is also regulated by pituitary-derived GH filtered from the blood compartment. Podocyte transcripts for IGF-1, IGF-1R, and GHR appear to be down-regulated in allografts, although this did not reach statistical significance (refer to FIG. 23). In contrast, fibroblasts express high levels of IGF-1 transcript in normal kidneys, which are significantly upregulated in allografts (refer to FIG. 23).

FIG. 22 shows a heatmap depicting a comparison for IGF pathway components between allograft cells versus normal kidney cells. Cell types are shown at bottom and top. Calibration of the fold difference is shown at right with blue designating decreased transcript expression in allografts and red designating increased transcript expression in allografts. Absence of color denotes transcript not detectably expressed in either normal kidney or allograft. Statistical significance (P<0.05) is shown by an asterisk. IGF-1 transcript expression is decreased in podocytes but does not reach statistical significance. In contrast, IGF-1 transcript is markedly and statistically increased in fibroblasts. IGF-1R transcript expression is decreased in most cell types. GHR expression is decreased in many cell types, but does not reach statistical significance in podocytes. IGFBP transcript expression is cell specific and is significantly up or down-regulated in different cell types.

FIG. 23A-23B show analysis of upregulated pathways in fibroblasts based on DEG between surveillance biopsies and healthy live donor kidneys using gene oncology. FIG. 23A shows Gene Ontology (GO) identifies increased expression of genes associated with IGF-1 signaling. FIG. 23B shows enriched genes in select upregulated pathways. This includes high expression of genes now identified as drivers of interstitial fibrosis (e.g., POSTN), increased oxidative phosphorylation, genes related to the collagen-containing extracellular matrix (ECM). Notably, this increased pro-fibrotic gene expression was observed in stable surveillance transplant biopsies with no detectable histologic abnormality.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a compound” is a reference to one or more compounds and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including human and non-human animals. In some embodiments, the subject is a vertebrate. In some embodiments, the subject is a mammal. In some embodiments, the subject is a human. As used herein, the term “preventing” refers to prophylactic steps taken to reduce the likelihood of a subject (e.g., an at-risk subject) from developing or suffering from a particular disease, disorder, or condition. The likelihood of the disease, disorder, or condition occurring in the subject need not be reduced to zero for the preventing to occur; rather, if the steps reduce the risk of a disease, disorder or condition across a population, then the steps prevent the disease, disorder, or condition for an individual subject within the scope and meaning herein.

As used herein, the term “treatment” (also “treat” or “treating”) refer to obtaining a desired pharmacologic and/or physiologic effect against a particular disease, disorder, or condition. Preferably, the effect is therapeutic, i.e., the effect partially or completely alleviates, ameliorates, relives, inhibits, delays onset of, reduces severity of, and/or reduces frequency, incidence or severity of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used herein, the terms “administration” and “administering” refer to the act of introducing a substance, such as a drug, prodrug, or other agent, or therapeutic treatment to a subject or in vivo, in vitro, or ex vivo cells, tissues, and organs. In general, any route of administration may be utilized including, for example, parenteral (e.g., intravenous), oral, topical, subcutaneous, peritoneal, intra-arterial, inhalation, vaginal, rectal, nasal, introduction into the cerebrospinal fluid, infusion, or instillation into body compartments. Exemplary routes of administration to the human body can be by parenteral administration (e.g., intravenously, subcutaneously, etc.), orally, etc.

As used herein, the term “approximately” and “about” is intended to encompass normal statistical variation as would be understood by those of ordinary skill in the art as appropriate to the relevant context. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would 15 exceed 100% of a possible value).

As used herein, the terms “co-administration” and “co-administering” refer to the administration of at least two agent(s) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent (e.g. an inhibitor of IGF-1R signaling and/or an inhibitor of GHR signaling) with a pharmaceutically acceptable carrier, inert or active, making the composition especially suitable for diagnostic or therapeutic use in vitro, in vivo or ex vivo. As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15^{th} Ed., Mack Publ. Co., Easton, Pa. (1975), incorporated herein by reference in its entirety.

The term “pharmaceutically acceptable” as used herein, refers to compositions that do not substantially produce adverse reactions, e.g., toxic, allergic, or immunological reactions, when administered to a subject.

### DETAILED DESCRIPTION

In some aspects, provided herein method of extending lifespan of a transplant. In some embodiments, provided herein are methods of extending lifespan of a transplant comprising providing an inhibitor of insulin-like growth factor 1 receptor (IGF-1R) signaling and/or an inhibitor of growth hormone receptor (GHR) signaling to a subject before, during, and/or after a transplant in the subject.

The methods described herein may be used to extend the lifespan of any suitable transplant. In some embodiments, the transplant is a solid organ transplant. In some embodiments, the transplant is a solid organ transplant selected from a kidney transplant, a liver transplant, a heart transplant, a lung transplant, a pancreas transplant, and an intestine transplant. In some embodiments, the transplant is a kidney transplant, a lung transplant, or a heart transplant. In some embodiments, the transplant is a kidney transplant. In some embodiments, the transplant is obtained from a living donor. In some embodiments, the transplant is obtained from a deceased donor.

In some aspects, provided herein are methods of determining the potential lifespan of a kidney transplant in a subject. The term “potential lifespan” as used herein refers to the predicted lifespan of a transplant, should a transplant be performed in a subject. In some embodiments, the methods of determining the potential lifespan of a kidney transplant in a subject comprise determining the effective kidney dose (eKD) of the kidney transplant, determining levels of IGF-1 in a sample obtained from the subject, and determining the potential lifespan of the kidney transplant based upon the eKD and/or levels of IGF-1. In some embodiments, the eKD of the kidney transplant is calculated as the donor: recipient body surface area ratio. In some embodiments, the eKD of the kidney transplant is calculated as the ratio of donor nephron mass:to recipient body weight. In some embodiments, methods of determining the potential lifespan of a kidney transplant further comprise determining one or more factors, such as the age of the subject receiving the transplant, the race of the subject receiving the transplant, the age of the donor (e.g. the source of the donor organ), and status of the donor (e.g. whether the donor is a living or deceased donor). In some embodiments, the one or more factors are used in the determination of potential lifespan of the kidney transplant in a subject.

In some embodiments, the methods comprise determining whether the kidney transplant (e.g. kidney allograft) has an acceptable lifespan or a poor lifespan based upon the eKD and/or levels of IGF-1. As used herein, the term “acceptable lifespan” is used in the broadest sense and indicates that the potential lifespan of the kidney transplant is sufficient such that methods to extend the lifespan of the kidney transplant involving providing an inhibitor of insulin-like growth factor 1 receptor (IGF-1R) signaling and/or an inhibitor of growth hormone receptor (GHR) signaling as described herein need not be performed in conjunction with the transplant. In contrast, a “poor lifespan” indicates that the potential lifespan of the kidney transplant is not sufficient, and methods to extend the lifespan of the kidney transplant involving providing an inhibitor of insulin-like growth factor 1 receptor (IGF-1R) signaling and/or an inhibitor of growth hormone receptor (GHR) signaling to the subject as described herein are recommended in conjunction with the transplant.

In some embodiments, whether the kidney transplant is determined to have an acceptable lifespan or a poor lifespan depends on the age of the subject, the current quality of life of the subject, the life expectancy of the subject, the expected improvement to quality of life of the subject should the kidney transplant be performed, and/or the expected increase life expectancy of the subject should the kidney transplant be performed. For example, a kidney transplant may be determined to have an “acceptable lifespan” if the expected improvement in quality of life and/or expected increase in life expectancy of the subject should the kidney transplant be performed be determined to be of sufficient benefit to the subject without taking extra steps to extend the lifespan of the kidney transplant (e.g. by providing an inhibitor of insulin-like growth factor 1 receptor (IGF-1R) signaling and/or an inhibitor of growth hormone receptor (GHR) signaling to the subject, as described herein). In some embodiments, a kidney transplant is determined to have an “acceptable lifespan” if the kidney transplant has a high probability (e.g. more than a 50% chance) of surviving for a sufficient duration of time in the subject. In contrast, in some embodiments a “poor lifespan” of the kidney transplant indicates that the transplant has a high probability of failing within a relatively short window of time relative to the lifespan of the subject. In some embodiments, transplant failure is indicated by one or more symptoms selected from hypertension, lower extremity edema, fatigue, fever, graft tenderness, uremia, oliguria, nausea, vomiting, a metallic taste, pericardial friction rub, asterixis, and/or a return to dialysis or another form of renal replacement, including re-transplantation.

In some embodiments, an “acceptable lifespan” of a kidney transplant for an older subject or a subject who is generally in worse condition (e.g. poorer health) is different from an “acceptable lifespan” for a subject of younger age or in better condition (e.g. better health). For example, in subjects 65 years of age of older, a kidney transplant may be determined to have an “acceptable lifespan” if the kidney transplant is predicted to have at least a 50% chance of surviving at least 5 years after transplant. However, for younger subjects, such as subjects less than 30 years of age, a kidney transplant predicted to only have a 50% chance of surviving at least 5 years after transplant may be determined to have a “poor lifespan”.

In some embodiments, for subjects 65 years of age of older, a kidney transplant may be determined to have an “acceptable lifespan” if the kidney transplant is predicted to have at least a 50% chance of surviving at least 5 years after transplant. In some embodiments, for subjects 65 years of age or older, a kidney transplant may be determined to have a “poor lifespan” if the kidney transplant is predicted to have at least a 50% chance of failing within the first 5 years after transplant.

In some embodiments, for subjects aged 51-65 years of age, a kidney transplant may be determined to have an “acceptable lifespan” if the kidney transplant is predicted to have at least a 50% chance of surviving at least 8 years after transplant. In some embodiments, for subjects 51-65 years of age, a kidney transplant may be determined to have a “poor lifespan” if the kidney transplant is predicted to have at least a 50% chance of failing within the first 8 years after transplant.

In some embodiments, for subjects aged 41-50 years of age, a kidney transplant may be determined to have an “acceptable lifespan” if the kidney transplant has at least a 50% chance of surviving at least 10 years after transplant. In some embodiments, for subjects aged 40-50 years of age, a kidney transplant may be determined to have a “poor lifespan” if the kidney transplant is predicted to have at least a 50% chance of failing within the first 10 years after transplant.

In some embodiments, for subjects aged 30-40 years of age, a kidney transplant may be determined to have an “acceptable lifespan” if the kidney transplant has at least a 50% chance of surviving at least 12 years after transplant. In some embodiments, for subjects aged 40-50 years of age, a kidney transplant may be determined to have a “poor lifespan” if the kidney transplant is predicted to have at least a 50% chance of failing within the first 12 years after transplant.

In some embodiments, for subjects less than 30 years of age, a kidney transplant may be determined to have an “acceptable lifespan” if the kidney transplant has at least a 50% chance of surviving at least 15 years after transplant. In some embodiments, for subjects less than 30 years of age, a kidney transplant may be determined to have a “poor lifespan” if the kidney transplant is predicted to have at least a 50% chance of failing within the first 15 years after transplant. In some embodiments, the methods comprise determining an acceptable lifespan of the kidney transplant when the eKD is equal to or above a threshold value. In some embodiments, the methods comprise determining an acceptable lifespan of the kidney transplant when the levels of IGF-1 in the sample are below a threshold value.

In some embodiments, the methods comprise determining an acceptable lifespan of the kidney transplant when the eKD is above 1.36. In some embodiments, the methods comprise determining an acceptable lifespan of the kidney transplant when the eKD is above 1 and the subject is at least 20 years old. In some embodiments, the methods comprise determining an acceptable lifespan of the kidney transplant when the eKD is above 1 and the subject is at least 30 years old. In some embodiments, the methods comprise determining an acceptable lifespan of the kidney transplant when the eKD is above 1 and the subject is at least 35 years old. In some embodiments, the methods comprise determining an acceptable lifespan of the kidney transplant when the eKD is above 0.75 and the subject is at least 40 years old.

In some embodiments, the methods comprise determining an acceptable lifespan of the kidney transplant when the levels of IGF-1 in the sample are below (e.g. less than) a threshold value of 92 ng/mL and the subject is at least 40 years old.

In some embodiments, the methods comprise determining a poor lifespan of the kidney transplant in the subject when the levels of IGF-1 in the sample are equal to or above a threshold value and the eKD is below a threshold value. In some embodiments, the methods comprise determining a poor lifespan of the kidney transplant when the levels of IGF-1 in the sample are equal to or above a threshold value of 400 ng/mL and the eKD is below a threshold value of 0.60. In some embodiments, the methods comprise determining a poor lifespan of the kidney transplant when the levels of IGF-1 in the sample are equal to or above a threshold value of 400 ng/ml and the eKD is below a threshold value of 0.50.

In some embodiments, the methods comprise determining a poor lifespan of the kidney transplant when the levels of IGF-1 in the sample are equal to or above a threshold value of 400 ng/mL, the donor is deceased, and the eKD is below a threshold value of 0.75. In some embodiments, the methods comprise determining a poor lifespan of the kidney transplant when the levels of IGF-1 in the sample are equal to or above a threshold value of 400 ng/mL, the donor is deceased, the eKD is below a threshold value of 0.75, and the subject is less than 50 years old.

In some embodiments, the methods comprise determining a poor lifespan of the kidney transplant when the levels of IGF-1 in the sample are equal to or above a threshold value of 400 ng/mL, the donor is deceased, the eKD is below a threshold value of 0.9, and the subject is less than 30 years old.

In some embodiments, the methods comprise determining a poor lifespan of the kidney transplant when the levels of IGF-1 in the sample are equal to or above a threshold value of 180 ng/mL, the donor is deceased, and the eKD is below a threshold value of 0.5.

In some embodiments, the methods comprise determining a poor lifespan of the kidney transplant when the levels of IGF-1 in the sample are equal to or above a threshold value of 180 ng/mL, the donor is deceased, the eKD is below a threshold value of 0.75, and the subject is less than 45 years old.

In some embodiments, the methods comprise determining a poor lifespan of the kidney transplant when the levels of IGF-1 in the sample are equal to or above a threshold value of 180 ng/mL, the donor is deceased, the eKD is below a threshold value of 0.9, and the subject is less than 30 years old.

In some embodiments, the methods comprise determining a poor lifespan of the kidney transplant when the levels of IGF-1 in the sample are equal to or above a threshold value of 92 ng/mL, the donor is deceased, the eKD is below a threshold value of 0.5, and the subject is less than 45 years old.

In some embodiments, the methods comprise determining a poor lifespan of the kidney transplant when the levels of IGF-1 in the sample are equal to or above a threshold value of 92 ng/mL, the donor is deceased, the eKD is below a threshold value of 1.36, and the subject is less than 30 years old.

In some embodiments, the methods comprise determining a poor lifespan of the kidney transplant when the levels of IGF-1 in the sample are equal to or above a threshold value of 400 ng/mL, the donor is living, and the eKD is below a threshold value of 0.6. In some embodiments, the methods comprise determining a poor lifespan of the kidney transplant when the levels of IGF-1 in the sample are equal to or above a threshold value of 400 ng/mL, the donor is living, the eKD is below a threshold value of 0.6, and the subject is less than 65 years old. In some embodiments, the methods comprise determining a poor lifespan of the kidney transplant when the levels of IGF-1 in the sample are equal to or above a threshold value of 400 ng/mL, the donor is living, the eKD is below a threshold value of 0.6, and the subject is less than 60 years old. In some embodiments, the methods comprise determining a poor lifespan of the kidney transplant when the levels of IGF-1 in the sample are equal to or above a threshold value of 400 ng/mL, the donor is living, the eKD is below a threshold value of 0.6, and the subject is less than 55 years old.

In some embodiments, the methods comprise determining a poor lifespan of the kidney transplant when the levels of IGF-1 in the sample are equal to or above a threshold value of 400 ng/mL, the donor is living, the eKD is below a threshold value of 0.7, and the subject is less than 30 years old. In some embodiments, the methods comprise determining a poor lifespan of the kidney transplant when the levels of IGF-1 in the sample are equal to or above a threshold value of 400 ng/mL, the donor is living, the eKD is below a threshold value of 0.6, and the subject is less than 25 years old.

In some embodiments, the methods comprise determining a poor lifespan of the kidney transplant when the levels of IGF-1 in the sample are equal to or above a threshold value of 180 ng/mL, the donor is living, the eKD is below a threshold value of 0.5, and the subject is less than 30 years old. In some embodiments, the methods comprise determining a poor lifespan of the kidney transplant when the levels of IGF-1 in the sample are equal to or above a threshold value of 180 ng/mL, the donor is living, the eKD is below a threshold value of 0.5, and the subject is less than 25 years old.

In some the method further comprises extending the lifespan of the kidney transplant, as described herein (e.g.when a poor lifespan of the kidney transplant is determined). For example, in some embodiments the method further comprises extending the lifespan of the kidney transplant by providing an inhibitor of insulin-like growth factor 1 receptor (IGF-1R) signaling and/or an inhibitor of growth hormone receptor (GHR) signaling to the subject when a poor lifespan of the kidney transplant is determined.

In some embodiments, the inhibitor of IGF-1R signaling and/or the inhibitor of GHR signaling is provided to the subject within 30 days of the transplant (e.g. no more than 30 days before receiving the transplant and no more than 30 days after receiving the transplant). The timespan of within 30 days of the transplant (e.g. within 30 days before or after the transplant) is referred to herein as the “peritransplant window” or the “peritransplant window”. The term “within” when used in reference to the transplant includes the time period before, during, and after the transplant. For example, providing the inhibitor of IGF-1R signaling and/or the inhibitor of GHR signaling to the subject “within” 30 days of the transplant indicates that the inhibitor may be provided to the subject anytime within the window of 30 days prior to 30 days after the transplant. In some embodiments, the inhibitor of IGF-1R signaling and/or the inhibitor of GHR signaling is provided to the subject within 3 weeks of the transplant. In some embodiments, the inhibitor of IGF-1R signaling and/or the inhibitor of GHR signaling is provided to the subject within 2 weeks of the transplant. In some embodiments, the inhibitor of IGF-1R signaling and/or the inhibitor of GHR signaling is provided to the subject within 10 days of the transplant. In some embodiments, the inhibitor of IGF-1R signaling and/or the inhibitor of GHR signaling is provided to the subject within 7 days of the transplant. For example, in some embodiments the inhibitor of IGF-1R signaling and/or the inhibitor of GHR signaling is provided to the subject within 30 days, within 28 days, within 26 days, within 24 days, within 22 days, within 20 days, within 18 days, within 16 days, within 14 days, within 12 days, within 10 days, within 9 days, within 8 days, within 7 days, within 6 days, within 5 days, within 4 days, within 3 days, within 2 days, or within 24 hours of transplant. In some embodiments, the inhibitor of IGF-1R signaling and/or the inhibitor of GHR signaling is provided to the subject within 24 hours, within 12 hours, within 6 hours, within 3 hours, or within 1 hour of the transplant.

In some embodiments, the inhibitor of IGF-1R signaling and/or the inhibitor of GHR signaling is provided to the subject in one or more doses occurring within a window from 30 days before to 30 days after the transplant. In some embodiments, the inhibitor of IGF-1R signaling and/or the inhibitor of GHR signaling is provided to the subject in one or more doses occurring within a window from 3 weeks before to 3 weeks after the transplant. In some embodiments, the inhibitor of IGF-1R signaling and/or the inhibitor of GHR signaling is provided to the subject in one or more doses occurring within a window from 2 weeks before to 2 weeks after the transplant. In some embodiments, the inhibitor of IGF-1R signaling and/or the inhibitor of GHR signaling is provided to the subject in one or more doses occurring within a window from 10 days before to 10 days after the transplant. In some embodiments, the inhibitor of IGF-1R signaling and/or the inhibitor of GHR signaling is provided to the subject in one or more doses occurring within a window from 7 days before to 7 days after the transplant. In some embodiments, the inhibitor of IGF-1R signaling and/or the inhibitor of GHR signaling is provided to the subject in one or more doses occurring within a window from 5 days before to 5 days after the transplant. In some embodiments, the inhibitor of IGF-1R signaling and/or the inhibitor of GHR signaling is provided to the subject in one or more doses occurring within a window from 3 days before to 3 days after the transplant. In some embodiments, the inhibitor of IGF-1R signaling and/or the inhibitor of GHR signaling is provided to the subject in one or more doses occurring within a window from 2 days before to 2 days after the transplant.

In some embodiments, the inhibitor of IGF-1R signaling and/or the inhibitor of GHR signaling is provided to the subject in one or more doses occurring within a window from 3 weeks before to 30 days after the transplant. In some embodiments, the inhibitor of IGF-1R signaling and/or the inhibitor of GHR signaling is provided to the subject in one or more doses occurring within a window from 3 weeks before to 3 weeks after the transplant. In some embodiments, the inhibitor of IGF-1R signaling and/or the inhibitor of GHR signaling is provided to the subject in one or more doses occurring within a window from 3 weeks before to 2 weeks after the transplant. In some embodiments, the inhibitor of IGF-1R signaling and/or the inhibitor of GHR signaling is provided to the subject in one or more doses occurring within a window from 3 weeks before to 10 days after the transplant. In some embodiments, the inhibitor of IGF-1R signaling and/or the inhibitor of GHR signaling is provided to the subject in one or more doses occurring within a window from 3 weeks before to 7 days after the transplant. In some embodiments, the inhibitor of IGF-1R signaling and/or the inhibitor of GHR signaling is provided to the subject in one or more doses occurring within a window from 3 weeks before to 5 days after the transplant. In some embodiments, the inhibitor of IGF-1R signaling and/or the inhibitor of GHR signaling is provided to the subject in one or more doses occurring within a window from 3 weeks before to 3 days after the transplant.

In some embodiments, the inhibitor of IGF-1R signaling and/or the inhibitor of GHR signaling is provided to the subject in one or more doses occurring within a window from 2 weeks before to 30 days after the transplant. In some embodiments, the inhibitor of IGF-1R signaling and/or the inhibitor of GHR signaling is provided to the subject in one or more doses occurring within a window from 2 weeks before to 3 weeks after the transplant. In some embodiments, the inhibitor of IGF-1R signaling and/or the inhibitor of GHR signaling is provided to the subject in one or more doses occurring within a window from 2 weeks before to 2 weeks after the transplant. In some embodiments, the inhibitor of IGF-1R signaling and/or the inhibitor of GHR signaling is provided to the subject in one or more doses occurring within a window from 2 weeks before to 10 days after the transplant. In some embodiments, the inhibitor of IGF-1R signaling and/or the inhibitor of GHR signaling is provided to the subject in one or more doses occurring within a window from 2 weeks before to 7 days after the transplant. In some embodiments, the inhibitor of IGF-1R signaling and/or the inhibitor of GHR signaling is provided to the subject in one or more doses occurring within a window from 2 weeks before to 5 days after the transplant. In some embodiments, the inhibitor of IGF-1R signaling and/or the inhibitor of GHR signaling is provided to the subject in one or more doses occurring within a window from 2 weeks before to 3 days after the transplant.

In some embodiments, the inhibitor of IGF-1R signaling and/or the inhibitor of GHR signaling is provided to the subject in one or more doses occurring within a window from 10 days before to 30 days after the transplant. In some embodiments, the inhibitor of IGF-1R signaling and/or the inhibitor of GHR signaling is provided to the subject in one or more doses occurring within a window from 10 days before to 3 weeks after the transplant. In some embodiments, the inhibitor of IGF-1R signaling and/or the inhibitor of GHR signaling is provided to the subject in one or more doses occurring within a window from 10 days before to 2 weeks after the transplant. In some embodiments, the inhibitor of IGF-1R signaling and/or the inhibitor of GHR signaling is provided to the subject in one or more doses occurring within a window from 10 days before to 10 days after the transplant. In some embodiments, the inhibitor of IGF-1R signaling and/or the inhibitor of GHR signaling is provided to the subject in one or more doses occurring within a window from 10 days before to 7 days after the transplant. In some embodiments, the inhibitor of IGF-1R signaling and/or the inhibitor of GHR signaling is provided to the subject in one or more doses occurring within a window from 10 days before to 5 days after the transplant. In some embodiments, the inhibitor of IGF-1R signaling and/or the inhibitor of GHR signaling is provided to the subject in one or more doses occurring within a window from 10 days before to 3 days after the transplant.

In some embodiments, the inhibitor of IGF-1R signaling and/or the inhibitor of GHR signaling is provided to the subject in one or more doses occurring within a window from 7 days before to 30 days after the transplant. In some embodiments, the inhibitor of IGF-1R signaling and/or the inhibitor of GHR signaling is provided to the subject in one or more doses occurring within a window from 7 days before to 3 weeks after the transplant. In some embodiments, the inhibitor of IGF-1R signaling and/or the inhibitor of GHR signaling is provided to the subject in one or more doses occurring within a window from 7 days before to 2 weeks after the transplant. In some embodiments, the inhibitor of IGF-1R signaling and/or the inhibitor of GHR signaling is provided to the subject in one or more doses occurring within a window from 7 days before to 10 days after the transplant. In some embodiments, the inhibitor of IGF-1R signaling and/or the inhibitor of GHR signaling is provided to the subject in one or more doses occurring within a window from 7 days before to 7 days after the transplant. In some embodiments, the inhibitor of IGF-1R signaling and/or the inhibitor of GHR signaling is provided to the subject in one or more doses occurring within a window from 7 days before to 5 days after the transplant. In some embodiments, the inhibitor of IGF-1R signaling and/or the inhibitor of GHR signaling is provided to the subject in one or more doses occurring within a window from 7 days before to 3 days after the transplant.

In some embodiments, more than one dose of the inhibitor of IGF-1R signaling is provided to the subject. For example, in some embodiments at least one dose of the inhibitor of IGF-1R signaling is provided to the subject before (e.g. within 30 days before) the transplant, and at least one dose of the inhibitor of IGF-1R signaling is provided to the subject after (e.g. within 30 days after) the transplant, including administration within a window described above. In some embodiments, more than one dose of the inhibitor of IGF-1R signaling is provided to the subject before (e.g. within 30 days before) the transplant. In some embodiments, more than one dose of the inhibitor of IGF-1R signaling is provided to the subject after (e.g. within 30 days after) the transplant.

In some embodiments, more than one dose of the inhibitor of GHR signaling is provided to the subject. For example, in some embodiments at least one dose of the inhibitor of GHR signaling is provided to the subject before (e.g. within 30 days before) the transplant, and at least one dose of the inhibitor of GHR signaling is provided to the subject after (e.g. within 30 days after) the transplant, including administration within a window described above. In some embodiments, more than one dose of the inhibitor of GHR signaling is provided to the subject before (e.g. within 30 days before) the transplant. In some embodiments, more than one dose of the inhibitor of GHR signaling is provided to the subject after (e.g. within 30 days after) the transplant.

In some embodiments, an inhibitor of IGF-1R signaling is provided to the subject. In some embodiments, the inhibitor of IGF-1R signaling inhibits activation of IGF-1R, inhibits phosphorylation of IGF-1R, and/or inhibits binding of a ligand to IGF-1R. In some embodiments, the inhibitor of IGF-1R signaling comprises a small molecule inhibitor. Suitable small molecule inhibitors of IGF-1R signaling include, but are not limited to, Linsitinib, NVP-AEW541, GSK1904529A, NVP-ADW742, BMS-536924, Ceritinib, AG-1024, GSK1838705A, BMS-754807, PQ 401, Ginsenoside Rg5, Ceritinib dihydrochloride, XL228, Brigatinib, Picropodophyllin, AZD3463, Insulin Degludec, S961, and Nordihydroguaiaretic acid (NGDA). In some embodiments, the inhibitor of IGF-1R signaling comprises an antibody. For example, in some embodiments the inhibitor of IGF-1R comprises an antibody that binds to IGF-1R (i.e. an anti-IGF-1R antibody). In some embodiments, the antibody is selected from Teprotumumab, Dalotuzumab, Figitumumab, Ganitumab, Robatumumab, and Cixutumumab.

In some embodiments, a single inhibitor of IGF-1R signaling is provided to the subject. In some embodiments, more than one inhibitor of IGF-1R signaling is provided to the subject. For example, in some embodiments two or more small molecule inhibitors are provided to the subject. As another example, in some embodiments at least one small molecule inhibitor and at least one antibody are provided to the subject.

In some embodiments, an inhibitor of GHR signaling is provided to the subject. In some embodiments, inhibitor of GHR signaling is a protein or an antibody. In some embodiments, the inhibitor of GHR signaling is Pegvisomant.

In some embodiments, the subject is a human subject. In some embodiments, the subject is an adult subject. As used herein, the term “adult” refers to an adult human subject at least 18 years old. In some embodiments, the subject is a peripubertal subject. As used herein, the term “peripubertal” refers to a human subject close to the time of puberty, typically aged 13-18 years old. In some embodiments, the subject is a prepubertal subject. As used herein, the term “prepubertal” refers to a subject that has not yet experienced puberty. For example, in some embodiments a prepubertal subject is less than 13 years of age (e.g. aged 12 years, 11 years, 10 years, 9 years, 8 years, 7 years, 6 years, etc.).

In some embodiments, after providing the inhibitor of IGF-1R signaling and/or inhibitor of GHR signaling to the subject for a suitable duration of time, a long-term therapeutic agent is provided to the subject. As used herein, the term “long-term therapeutic agent” or “long-term therapeutic” refers to an agent suitable for delivery to the subject for an extended period of time beyond the peritransplant window. For example, in some embodiments the inhibitor of IGF-1R signaling and/or the inhibitor of GHR signaling is provided to the subject for a suitable number of doses within the peritransplant window (e.g. within 30 days of transplant), after which time a long-term therapeutic agent is provided to the subject. In some embodiments, the long-term therapeutic agent is provided to the subject in addition to the inhibitor of IGF-1R signaling and/or inhibitor of GHR signaling (in other words, treatment with the inhibitor of IGF-1R signaling and/or inhibitor of GHR signaling continues beyond the peritransplant window and is supplemented with a long-term therapeutic agent). In some embodiments, treatment with the inhibitor of IGF-1R signaling and/or inhibitor of GHR signaling is discontinued (e.g. ceases after the peritransplant window has expired).

For peripubertal subjects, the duration of delivery of the inhibitor of IGF-1R signaling and/or inhibitor of GHR signaling should be determined to effectively extend the lifespan of the transplant (e.g. the kidney transplant) while minimizing growth-restricting side effects of the inhibitor. Accordingly, in some embodiments for peripubertal subjects treatment with the inhibitor of IGF-1R signaling and/or GHR signaling ceases about 3 weeks after transplant. In some embodiments, treatment with the inhibitor of IGF-1R signaling and/or GHR signaling ceases about 3 weeks after transplant and is replaced by a long-term therapeutic agent. For example, in some embodiments the subject is a peripubertal subject and the method comprises providing to the subject one or more doses of an inhibitor of IGF-1R signaling and/or an inhibitor of GHR signaling within a window of from 30 days before to 21 days after transplant, followed by replacing the inhibitor of IGF-1R signaling and/or an inhibitor of GHR signaling with a long-term therapeutic agent.

In some embodiments, the long-term therapeutic agent is selected from an angiotensin 2 receptor inhibitor, an angiotensin converting enzyme (ACE) inhibitor, a sodium-glucose co-transporter 2 (SGLT2) inhibitor, and a Glucagon-like peptide-1 receptor agonist (GLP1RA).

In some embodiments, the transplant is a kidney transplant, and levels of IGF-1R in a sample obtained from the subject prior to kidney transplant are above a threshold value for IGF-1R and/or an effective kidney dose (eKD) of the kidney transplant is below a threshold value for eKD. In some embodiments, the eKD of the kidney transplant is calculated as the donor: recipient body surface area ratio. In some embodiments, the eKD of the kidney transplant is calculated as the ratio of donor nephron mass:to recipient body weight.

In some aspects, provided herein are methods of extending lifespan of a transplant wherein the donor organ is obtained from a deceased subject. In some embodiments, provided herein is a method of extending lifespan of a transplant, comprising contacting a donor organ obtained from a deceased subject with an inhibitor of insulin-like growth factor 1 receptor (IGF-1R) signaling and/or an inhibitor of growth hormone receptor (GHR) signaling prior to transplant of the donor organ into a subject. Donor organs from deceased subjects are more often likely to be rejected as non-viable due to physiological state of the organ. The methods described herein can be used to achieve successful transplant using organs that would have otherwise been discarded as non-viable, and are therefore useful for extending the donor pool of available organs for transplant into subjects in need. In some embodiments, contacting the donor organ with the inhibitor comprises perfusing the donor organ with a composition comprising the inhibitor of IGF-1R signaling and/or the inhibitor of GHR signaling. In some embodiments, the donor organ is contacted with (e.g. perfused with) an inhibitor of IGF-1R signaling. In some embodiments, the inhibitor of IGF-1R signaling inhibits activation of IGF-1R, inhibits phosphorylation of IGF-1R, and/or inhibits binding of a ligand to IGF-1R. In some embodiments, the inhibitor of IGF-1R signaling comprises a small molecule inhibitor. Suitable small molecule inhibitors of IGF-1R signaling include, but are not limited to, Linsitinib, NVP-AEW541, GSK1904529A, NVP-ADW742, BMS-536924, Ceritinib, AG-1024, GSK1838705A, BMS-754807, PQ 401, Ginsenoside Rg5, Ceritinib dihydrochloride, XL228, Brigatinib, Picropodophyllin, AZD3463, Insulin Degludec, S961, and Nordihydroguaiaretic acid (NGDA). In some embodiments, the inhibitor of IGF-1R signaling comprises an antibody. For example, in some embodiments the inhibitor of IGF-1R comprises an antibody that binds to IGF-1R (i.e. an anti-IGF-1R antibody). In some embodiments, the antibody is selected from Teprotumumab, Dalotuzumab, Figitumumab, Ganitumab, Robatumumab, and Cixutumumab.

In some embodiments, the donor organ is contacted with a single inhibitor of IGF-1R signaling. In some embodiments, the donor organ is contacted with more than one inhibitor of IGF-1R signaling. For example, in some embodiments the donor organ is contacted with two or more small molecule inhibitors. As another example, in some embodiments the donor organ is contacted with at least one small molecule inhibitor and at least one antibody.

In some embodiments, the donor organ is contacted with an inhibitor of GHR signaling. In some embodiments, inhibitor of GHR signaling is a protein or an antibody. In some embodiments, the inhibitor of GHR signaling is Pegvisomant.

In some embodiments, the donor organ is contacted with (e.g. perfused with) the inhibitor of IGF-1R signaling and/or the inhibitor of GHR signaling proximal to the time of transplanting the donor organ into a subject. For example, in some embodiments the donor organ is perfused with the inhibitor of IGF-1R signaling and/or the inhibitor of GHR signaling less than 24 hours prior to the donor organ being transplanted into the subject (e.g. less than 24 hours, less than 23 hours, less than 22 hours, less than 21 hours, less than 20 hours, less than 19 hours, less than 18 hours, less than 17 hours, less than 16 hours, less than 15 hours, less than 14 hours, less than 13 hours, less than 12 hours, less than 11 hours, less than 10 hours, less than 9 hours, less than 8 hours, less than 7 hours, less than 6 hours, less than 5 hours, less than 4 hours, less than 3 hours, less than 2 hours, or within 1 hour of transplant).

In some aspects, provided herein are methods of extending kidney lifespan in a subject having kidney dysfunction. In some embodiments, methods of extending kidney lifespan in a subject having kidney dysfunction comprise providing an inhibitor of insulin-like growth factor 1 receptor (IGF-1R) signaling and/or an inhibitor of growth hormone receptor (GHR) signaling to a subject having kidney dysfunction.

In some embodiments, the kidney dysfunction is selected from hyperfiltration, glomerular injury, reduced nephron number, and glomerular disease. In some embodiments, the kidney dysfunction is hyperfiltration. In some embodiments, the hyperfiltration is induced by hypertension, partial nephrectomy, kidney transplantation, chronic kidney disease, diabetes, and/or obesity. In some embodiments, the kidney dysfunction is glomerular disease. Suitable glomerular diseases include, but are not limited to, focal segmental glomerulosclerosis (GSGS), acute glomerulonephritis, crescentic glomerulonephritis, IgA nephropathy, lupus nephritis, cryoglobulinemia, Membranoproliferative glomerulonephritis (MPGN) transplant glomerulopathy, membranous glomerulopathy, Genetic glomerular disease, and minimal change disease.

In some embodiments, an inhibitor of IGF-1R signaling is provided to the subject. In some embodiments, the inhibitor of IGF-1R signaling inhibits activation of IGF-1R, inhibits phosphorylation of IGF-1R, and/or inhibits binding of a ligand to IGF-1R. In some embodiments, the inhibitor of IGF-1R signaling comprises a small molecule inhibitor. Suitable small molecule inhibitors of IGF-1R signaling include, but are not limited to, Linsitinib, NVP-AEW541, GSK1904529A, NVP-ADW742, BMS-536924, Ceritinib, AG-1024, GSK1838705A, BMS-754807, PQ 401, Ginsenoside Rg5, Ceritinib dihydrochloride, XL228, Brigatinib, Picropodophyllin, AZD3463, Insulin Degludec, S961, and Nordihydroguaiaretic acid (NGDA). In some embodiments, the inhibitor of IGF-1R signaling comprises an antibody. For example, in some embodiments the inhibitor of IGF-1R comprises an antibody that binds to IGF-1R (i.e. an anti-IGF-1R antibody). In some embodiments, the antibody is selected from Teprotumumab, Dalotuzumab, Figitumumab, Ganitumab, Robatumumab, and Cixutumumab.

In some embodiments, a single inhibitor of IGF-1R signaling is provided to the subject. In some embodiments, more than one inhibitor of IGF-1R signaling is provided to the subject. For example, in some embodiments two or more small molecule inhibitors are provided to the subject. As another example, in some embodiments at least one small molecule inhibitor and at least one antibody are provided to the subject.

In some embodiments, an inhibitor of GHR signaling is provided to the subject. In some embodiments, inhibitor of GHR signaling is a protein or an antibody. In some embodiments, the inhibitor of GHR signaling is Pegvisomant.

For any of the embodiments described herein involving providing an inhibitor of IGF-1R signaling and/or an inhibitor of GHR signaling to a subject, the inhibitor may be provided to the subject by any suitable route of administration, including parenteral (e.g. by injection, including subcutaneous, intramuscular, intravenous, intrathecal, intraarterial, etc.) and oral routes. The appropriate dose of the inhibitor of IGF-1R signaling and/or inhibitor of GHR signaling depends various factors, including the specific inhibitor being used, the age of the subject, the weight of the subject, and the general health of the subject. Generally speaking, an appropriate dose ranges from 1 ng/kg to 100 mg/kg body weight. In some embodiments, the dose of the inhibitor is 1 ng/kg to 100 mg/kg, 100 ng/kg to 90 mg/kg, 500 ng/kg to 80 mg/kg, lug/kg to 70 mg/kg, 100 μg/kg to 60 mg/kg, 200 μg/kg to 50 mg/kg, 300 μg/kg to 40 mg/kg, 400 μg/kg to 30 mg/kg, 500 μg/kg to 20 mg/kg, 600 μg/kg to 15 mg/kg, 700 μg/kg to 10 mg/kg, 800 μg/kg to 5 mg/kg, 900 μg/kg to 2.5 mg/kg, or about 1 mg/kg.

### EXAMPLES

### Example 1

Insulin-like growth factor-1 (IGF-1) was identified herein as a driver of shorter kidney allograft longevity. In this example, median allograft longevity (death-censored survival) for every recipient aged 0-60 years was calculated using the Organ Procurement and Transplantation Network database (n=366,404). The average IGF-1 level for each recipient age group that increases to a peak at puberty and declines thereafter into older age was imputed from a normal reference population. A strong relationship was observed between allograft longevity and imputed IGF-1 level, indicating that pre-transplant IGF-1 level is related to allograft longevity. IGF-1 interacted with the kidney dose in relation to death-censored graft failure, (HR=1.87, p=0.01), proteinuria (HR=2.11, p=0.01) and alloimmune response (HR 1.29, p<0.05). Podocytes express high level transcript for growth hormone receptor, IGF-1, IGF-1 receptor as components of an intrinsic IGF-1 regulatory system. Fibroblasts normally express high level IGF-1 transcript that is further amplified in allografts.

Pre-transplant IGF-1 levels in stored serum samples from consecutive allograft recipients with a median 8.6-year follow-up (n=216) were measured. The estimated Kidney Dose (eKD) transplanted was calculated as the donor: recipient body surface area ratio, a surrogate for donor nephron mass in relation to recipient body size, that determines the degree of allograft hyperfiltration. Adjusted Cox models demonstrated a significant interaction between pre-transplant IGF-1 level and eKD that was related to allograft longevity (p=0.007), post-transplant proteinuria (p=0.007), and composite allo-immune response (biopsy-proven acute rejection or de novo donor-specific antibodies) (p=0.049). Increased T-Cell medicated rejection grade Banff 1A and above drove the allo-immune response.

Taken together, the data presented herein indicate that allograft longevity is determined at least in part by the amount of IGF-1 hyperfiltered from the blood into the allograft at transplantation, and that IGF-1 signaling plays a role in allograft longevity.

### Results

### Population-Level Data

Descriptive characteristics of the OPTN cohort: After applying the inclusion and exclusion criteria, 366,404 kidney-only transplant recipients aged 0-60 years were available for analysis. Table 1 shows the clinical characteristics of donors, recipients, and transplantation factors for the cohort. The median allograft survival time for the cohort was 15.3 years, with 25% and 75% survival times of 8.5 to 24.1 years, respectively. A total of 104,209 allograft failures occurred during the period of observation.

TABLE 1
Donor, recipient, and transplant characteristics of
the OPTN cohort used for analysis (n = 366,404).

| Donor Factors | |
|---|---|
| Donor Age | 39.1 ± 12.6 |
| Donor Type | |
| Living | 141,584 (38.6%) |
| Deceased Non-ECD | 170,122 (46.4%) |
| Deceased ECD | 21,973 (6.0%) |
| Missing | 32,725 (8.9%) |
| Donor Gender | |
| Female | 171,171 (46.7%) |
| Male | 195,232 (53.3%) |
| Donor Race | |
| White | 260,011 (71.0%) |
| Black | 43,355 (11.8%) |
| Others or missing | 63,038 (17.2%) |
| Donor BMI (Missing % = 16.7) | 27.2 ± 5.8 |
| Donor BSA (Missing % = 16.6) | 1.94 ± 0.26 |
| Transplant Factors | |
| Cold Ischemia Factor | |
| <12 hours | 158,786 (43.3%) |
| >12 hours | 168,802 (46.1) |
| Missing | 38,816 (10.6%) |
| HLA Mismatch | |
| 0 | 31,214 (8.5%) |
| 1 | 15,993 (4.4%) |
| 2 | 38,366 (10.5%) |
| 3 | 73,301(20.0%) |
| 4 | 78,655 (21.5%) |
| 5 | 83,730 (22.9%) |
| 6 | 41,030 (11.2%) |
| Missing | 4,115 (1.1%) |
| Recipient Factors | |
| Recipient Age (years) | 41.0 ± 13.3 |
| Recipient BMI (Missing % = 4.7) | 26.20 ± 5.72 |
| Recipient Gender | |
| Female | 145,549 (39.7%) |
| Male | 220,855 (60.3%) |
| Recipient Race | |
| White | 193,721 (52.9%) |
| Black | 92,449 (25.2%) |
| Others | 80,234 (21.9%) |
| Recipient PRA | |
| PRA <20 | 245,415 (67.0%) |
| PRA >20 | 67,789 (18.5%) |
| Missing | 81,608 (14.5%) |
| Induction | |
| No | 74,902 (20.4%) |
| Yes | 291,502 (79.6%) |
| Maintenance | |
| CNI, MMF, Steroids | 236,772 (64.6%) |
| CNI, MMF | 62,667 (17.1%) |
| CNI, Steroids | 16,108 (4.4%) |
| CNI only | 2,673 (0.7%) |
| No CNI | 28,225 (7.7%) |
| Missing data | 19,959 (5.5%) |
| Delayed Graft Function | |
| No | 297,564 (81.2%) |
| Yes | 65,778 (17.9%) |
| Missing data | 3,062 (0.8%) |
| Previous Organ Transplant | |
| No | 319,526 (87.2%) |
| Yes | 23,134 (12.8%) |
| Dialysis Prior to Transplant | |
| No | 59,063 (16.1%) |
| Yes | 296,348 (8.9%) |
| Missing data | 10,993 (3.0%) |

BMI: Body Mass Index; CNI: Calcineurin Inhibitor; CIT: Cold Ischemia Time; DD: Deceased Donor; ECD: Expanded Criteria Donor; MMF: Mycophenolate Mofetil; PRA: Panel Reactive Antibodies. Data are shown as the mean with standard errors for continuous variables. Categorical variables are reported as percentages.

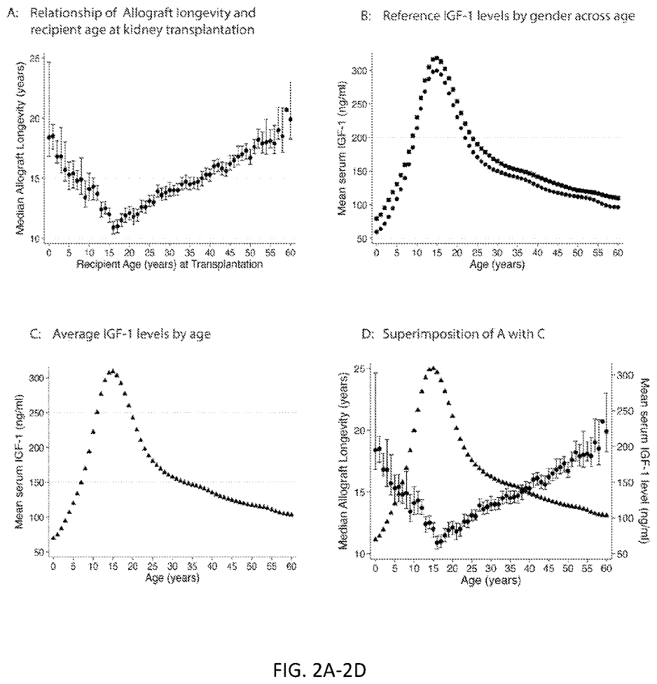

Relationship between recipient age and kidney allograft longevity. Kidney allograft longevity was shortest in the 16-year-old group (10.9 years) and longest in the 60-year-old age group (19.9 years) (FIG. 2A). Numerical data for allograft recipient longevity by age are shown in Table 8.

TABLE 8
Ppulation study. Median Deathcensored
graft failure by recipient age

| Age | N | Median DCGF | Std. Err | LCL | UCL |
|---|---|---|---|---|---|
| 0 | 97 | 18.4 | 1.3 | 16.8 | 24.6 |
| 1 | 950 | 18.5 | 0.5 | 17.4 | 19.5 |
| 2 | 1028 | 16.8 | 0.5 | 16.3 | 18.1 |
| 3 | 813 | 16.8 | 0.9 | 15.5 | 19.2 |
| 4 | 677 | 16.7 | 0.8 | 14.8 | 18.0 |
| 5 | 719 | 15.3 | 0.6 | 14.1 | 16.4 |
| 6 | 621 | 15.4 | 0.6 | 14.2 | 16.4 |
| 7 | 710 | 14.8 | 0.4 | 14.1 | 15.6 |
| 8 | 750 | 14.9 | 0.8 | 13.7 | 16.7 |
| 9 | 844 | 13.4 | 0.6 | 12.6 | 14.8 |
| 10 | 969 | 14.1 | 0.5 | 13.2 | 15.4 |
| 11 | 1007 | 14.3 | 0.4 | 13.2 | 15.0 |
| 12 | 1262 | 13.7 | 0.4 | 13.2 | 14.4 |
| 13 | 1470 | 12.4 | 0.3 | 11.7 | 13.0 |
| 14 | 1731 | 12.5 | 0.3 | 12.0 | 13.1 |
| 15 | 2058 | 12.0 | 0.3 | 11.4 | 12.5 |
| 16 | 2358 | 10.9 | 0.3 | 10.4 | 11.4 |
| 17 | 2675 | 11.0 | 0.2 | 10.5 | 11.5 |
| 18 | 2443 | 11.5 | 0.3 | 11.0 | 12.0 |
| 19 | 2194 | 11.9 | 0.3 | 11.3 | 12.5 |
| 20 | 2394 | 12.1 | 0.3 | 11.5 | 12.6 |
| 21 | 2717 | 11.8 | 0.3 | 11.1 | 12.5 |
| 22 | 3093 | 12.0 | 0.3 | 11.4 | 12.4 |
| 23 | 3473 | 12.6 | 0.3 | 12.2 | 13.2 |
| 24 | 3893 | 12.6 | 0.3 | 12.1 | 13.1 |
| 25 | 4250 | 13.1 | 0.2 | 12.6 | 13.6 |
| 26 | 4605 | 13.0 | 0.2 | 12.7 | 13.6 |
| 27 | 4899 | 13.9 | 0.2 | 13.4 | 14.4 |
| 28 | 5282 | 13.6 | 0.3 | 13.2 | 14.2 |
| 29 | 5670 | 13.8 | 0.2 | 13.2 | 14.3 |
| 30 | 5962 | 14.0 | 0.3 | 13.5 | 14.6 |
| 31 | 6145 | 14.0 | 0.2 | 13.6 | 14.4 |
| 32 | 6487 | 14.0 | 0.2 | 13.6 | 14.6 |
| 33 | 6638 | 14.5 | 0.3 | 14.0 | 15.1 |
| 34 | 7018 | 14.7 | 0.3 | 14.1 | 15.3 |
| 35 | 7091 | 14.5 | 0.3 | 14.0 | 15.1 |
| 36 | 7450 | 14.6 | 0.2 | 14.1 | 15.1 |
| 37 | 7878 | 14.7 | 0.2 | 14.1 | 15.0 |
| 38 | 8043 | 15.0 | 0.3 | 14.4 | 15.6 |
| 39 | 8353 | 15.3 | 0.2 | 14.9 | 15.8 |
| 40 | 8572 | 15.3 | 0.2 | 14.7 | 15.8 |
| 41 | 8901 | 16.0 | 0.3 | 15.4 | 16.5 |
| 42 | 9375 | 16.1 | 0.2 | 15.6 | 16.6 |
| 43 | 9320 | 15.8 | 0.3 | 15.3 | 16.4 |
| 44 | 9840 | 15.6 | 0.3 | 15.2 | 16.3 |
| 45 | 10068 | 16.2 | 0.3 | 15.7 | 16.9 |
| 46 | 10336 | 16.5 | 0.3 | 15.9 | 16.9 |
| 47 | 10574 | 16.8 | 0.3 | 16.1 | 17.7 |
| 48 | 10728 | 17.0 | 0.3 | 16.5 | 17.7 |
| 49 | 11200 | 17.3 | 0.3 | 16.6 | 17.9 |
| 50 | 11546 | 16.7 | 0.3 | 16.2 | 17.4 |
| 51 | 11574 | 17.6 | 0.4 | 17.0 | 18.2 |
| 52 | 11445 | 18.2 | 0.5 | 17.5 | 19.1 |
| 53 | 11672 | 17.9 | 0.4 | 17.1 | 18.8 |
| 54 | 11725 | 18.0 | 0.6 | 17.0 | 19.9 |
| 55 | 11813 | 18.1 | 0.5 | 17.5 | 19.1 |
| 56 | 11967 | 17.9 | 0.6 | 17.0 | 19.4 |
| 57 | 11845 | 19.0 | 0.7 | 18.3 | 20.9 |
| 58 | 11852 | 18.5 | 0.8 | 17.2 | 20.8 |
| 59 | 11870 | 20.7 | . | 19.0 | . |
| 60 | 11659 | 19.9 | 0.9 | 18.3 | 23.0 |

Variation of serum IGF-1 level across the human lifespan is inversely related to allograft longevity. FIG. 2B shows the distribution of average population IGF-1 values across ages in males (triangles) and females (circles), as reported by Bidlingmaier et al. (Bidlingmaier M, et al. Reference intervals for insulin-like growth factor-1 (igf-i) from birth to senescence: results from a multicenter study using a new automated chemiluminescence IGF-I immunoassay conforming to recent international recommendations. J Clin Endocrinol Metab. 2014; 99(5):1712-1721). Both curves peak at 16 years old, with female values slightly lower than males. FIG. 2C shows age-averaged values across the combined female and male populations. FIG. 2D (a composite of FIGS. 2A and 2C) demonstrates the inverse relationship between IGF-1 level at the time of transplantation and allograft longevity, with the IGF-1 curve peaking and the allograft longevity curve hitting its lowest point at 16 years old. This result indicates that IGF-1 level at transplantation is related to allograft longevity.

### Individualized Relationship Between Pre-Transplant IGF-1 Level and Allograft Outcome.

Table 2 shows transplant, donor, and recipient characteristics and pre-transplant IGF-1 and IGF-2 values for the Individual study cohort. Table 3 shows outcomes of primary interest (death-censored allograft survival) and secondary interest (proteinuria and a composite allo-immune score). Additional information is provided for subgroups of interest, including biopsy-proven rejection, T-cell-mediated rejection, and de novo donor-specific 5 antibody detection.

TABLE 2
Donor, recipient, and transplant characteristics of the University
of Michigan cohort used for analysis (n = 216).

| Recipient Characteristics at transplant | |
|---|---|
| Age | 47.4 ± 17.7 (1.75-77) |
| IGF-1 (ng/ml) | 206.8 ± 114.2 (16.3-624.6) |
| IGF-2 (ng/ml) (n = 187) | 1067.6 ± 565.8 (144-2655.6) |
| Race | |
| White | 153 (70.8%) |
| Black | 48 (22.2%) |
| Other | 15 (6.9%) |
| Gender | |
| Female | 81 (37.50%) |
| Male | 135 (62.50%) |
| Height (cm) | 168.4 ± 17.31 (75.4-193) |
| Weight (kg) | 84.1 ± 25.9 (10.3-143.7) |
| BSA | |
| Cause of kidney failure | |
| DM1 | 11 (5.09%) |
| DM2 | 58 (26.85%) |
| GN | 44 (20.37%) |
| HTN | 30 (13.89%) |
| PKD | 18 (8.33%) |
| OTH | 55 (25.46%) |
| Previous transplant | |
| Sensitization status | |
| cPRA1 | 0, IQR (0, 0) (range: 0-94) |
| cPRA2 | 0, IQR (0-0) (range: 0-91) |
| Induction | |
| None | 46 (21.3%) |
| Simulect | 9 (4.2%) |
| Thymoglobulin | 161 (74.5%) |
| Transplant and Organ Characteristics | |
| Organ Type | |
| Living Donor | 103 (47.7%) |
| Deceased Donor | 113 (52.3%) |
| Donation after cardiac death | |
| No | 80 (70.8% of deceased donors) |
| Yes | 33 (29.2% of deceased donors) |
| Cold ischemia time (hours) | 568 ± 484 (24-1877) |
| Donor: Recipient BSA ratio | 1.06 ± 0.45 (0.26-4.19) |
| Donor Characteristics at transplant | |
| Age (n = 215) | 40.2 ± 14.2 (2-83) |
| Race | |
| White | 183 (85.1%) |
| Black | 17 (7.9%) |
| Others | 15 (7%) |
| Gender | |
| Female | 115 (53.2%) |
| Male | 101 (46.8%) |
| Height (cm) | 169.71 ± 13.60 (76.2-195.6) |
| Weight (kg) | 81.41 ± 22.11 (11-158.8) |
| Donor BSA | 1.94 ± 0.31 (0.49-2.76) |
| Donor Hypertension | |
| No | 196 (91.2%) |
| Yes (n = 215) | 19 (8.8%) |
| Donor Diabetes | |
| No | 211 (97.7%) |
| Yes | 5 (2.3%) |

BSA: Body Surface Area; cPRA: Calculated panel reactive antibodies. DM1: Diabetes mellitus type 1; DM2: Diabetes mellitus type 2; GN: Glomerular disease; HTN: Hypertension; IGF-1: Insulin-like growth factor 1; IGF-2: Insulin-like growth factor 2; BSA: Body Surface Area; PKD: Polycystic kidney disease; OTH: Others, not in the above classification

TABLE 3
Primary or secondary endpoints reached in the follow-up period.

| Primary endpoint | Yes | No | % positive |
|---|---|---|---|
| Death-censored allograft failure | 42 | 174 | 19.4 |
| Secondary endpoint | | | |
| Post-transplant proteinuria (>1g/g creatinine) | 33 | 183 | 15.3 |
| Composite allo-immune response | 78 | 138 | 36.1 |
| Subgroups of clinical interest | | | |
| Biopsy-proven acute rejection (BPAR) | 44 | 171 | 20.5 |
| Rejection type amongst those with BPAR (n = 44) | | | |
| TCMR grade 1A and above | 29 | 15 | 65.9 |
| ABMR | 10 | 34 | 22.7 |
| Mixed rejection | 5 | 39 | 11.4 |
| De novo Donor-specific antibody (DSA) | 55 | 161 | 25.5 |
| DSA class amongst those with de novo DSA (n = 55) | | | |
| Class 1 only when first detected | 18 | 37 | 32.7 |
| Class 2 only when first detected | 29 | 26 | 52.7 |
| Both Class 1 and Class 2 when detected | 8 | 47 | 14.5 |

Table Legend: Composite of alloimmune response is the number who developed biopsy-proven acute rejection and/or any class of donor-specific antibody.
ABMR: Antibody Mediated Rejection.
DSA: Donor Specific Antibodies.
TCMR: T-Cell Mediated Rejection.
Note:
Biopsies were evaluated using Banff 2007 and the 2011 update as the standard at that point. DSA was only indicated as detected if the MFI was >1000.

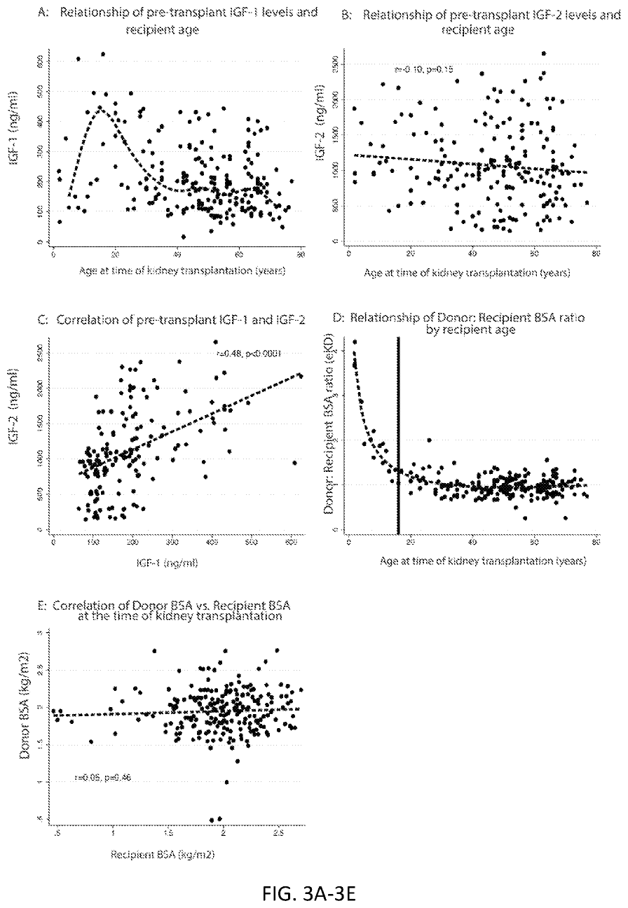

Relation of pre-transplant IGF-1 level to variables of relevance: FIG. 3A shows that the age-specific average IGF-1 values in the pre-transplant (uremic) population increased to peak at 16 years and declined into older age, comparable to age-specific average IGF-1 values in the general population (shown in FIG. 2C). No age-related effect was seen for IGF-2 (FIG. 3B), although IGF-1 and IGF-2 correlated moderately with each other (R=0.48, p<0.001) (FIG. 3C). FIG. 3D shows the BSA ratio (designated as the effective kidney dose [eKD]) delivered at transplantation across different ages in the cohort. Higher eKDs were delivered in the pre-pubertal group than in the post-pubertal group because of their relatively smaller body size in relation to the body size of the kidney donor. FIG. 3E shows no correlation between donor BSA and recipient BSA in the cohort, consistent with no intentional donor-to-recipient matching of kidney and body size occurring at the time of kidney transplantation in this cohort.

Relationship of IGF-1 levels at the time of transplantation to death-censored kidney allograft failure at different levels of kidney dose (eKD). It was investigated whether allograft IGF-1 exposure was a function of the interaction between the IGF-1 level and the degree of hyperfiltration (assessed as the donor: recipient BSA ratio). As shown in Table 4, the interaction between IGF-1 and the eKD was significantly related to allograft longevity (P=0.007).

TABLE 4
Multivariable cox model for death-
censored kidney allograft failure.

| | Hazard | Std. | | | 95% confidence | |
|---|---|---|---|---|---|---|
| | ratio | err. | z | P > z | interval | |
| IGF-1 level | 1.01 | 0.01 | 2.40 | 0.02 | 1.00 | 1.02 |
| eKD | 1.01 | 0.01 | 0.96 | 0.34 | 0.99 | 1.04 |
| Interaction term | 0.99 | 0.0001 | −2.70 | 0.007 | 0.99 | 0.99 |
| (IGF-1 * eKD) | | | | | | |

Other significantly related factors

| | | | | | | |
|---|---|---|---|---|---|---|
| Recipient age at TP | 0.96 | 0.01 | −2.82 | 0.005 | 0.94 | 0.99 |
| Recipient race | | | | | | |
| Black | 3.19 | 1.17 | 3.18 | 0.001 | 1.56 | 6.52 |
| Others | 1.97 | 1.48 | 0.91 | 0.36 | 0.46 | 8.55 |
| Male | 1.09 | 0.38 | 0.25 | 0.81 | 0.55 | 2.17 |
| Deceased donor | 3.68 | 1.48 | 3.25 | 0.001 | 1.66 | 8.11 |
| Donor age | 1.04 | 0.01 | 3.45 | 0.001 | 1.02 | 1.07 |
| Donor race | | | | | | |
| Black | 2.81 | 1.43 | 2.03 | 0.04 | 1.04 | 7.66 |
| Others | 3.12 | 1.62 | 2.19 | 0.03 | 1.13 | 8.64 |

Table legend:
BSA: Body Surface Area
D:R: Donor to Recipient
eKD: Effective Kidney Dose
IGF-1: Insulin-like growth factor 1
TP: Transplantation

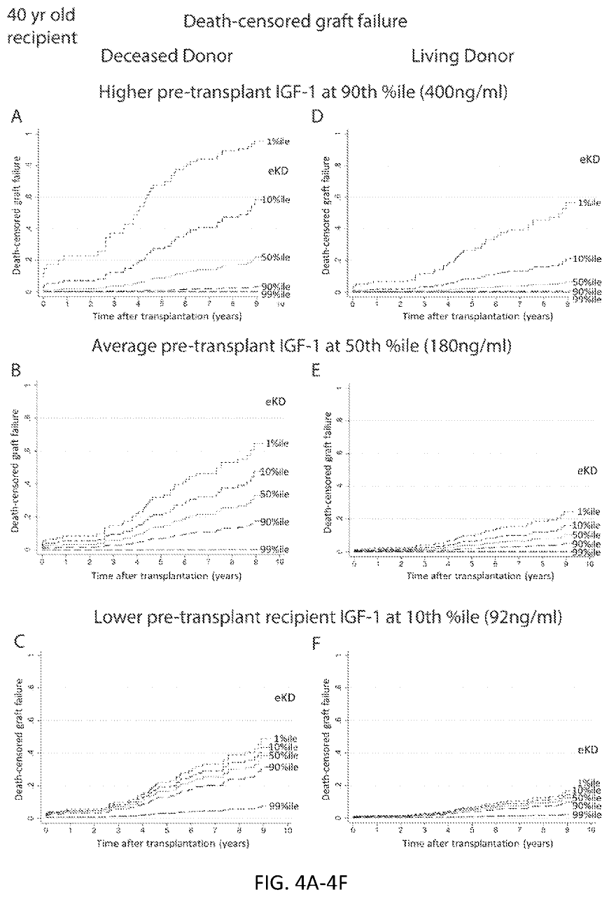

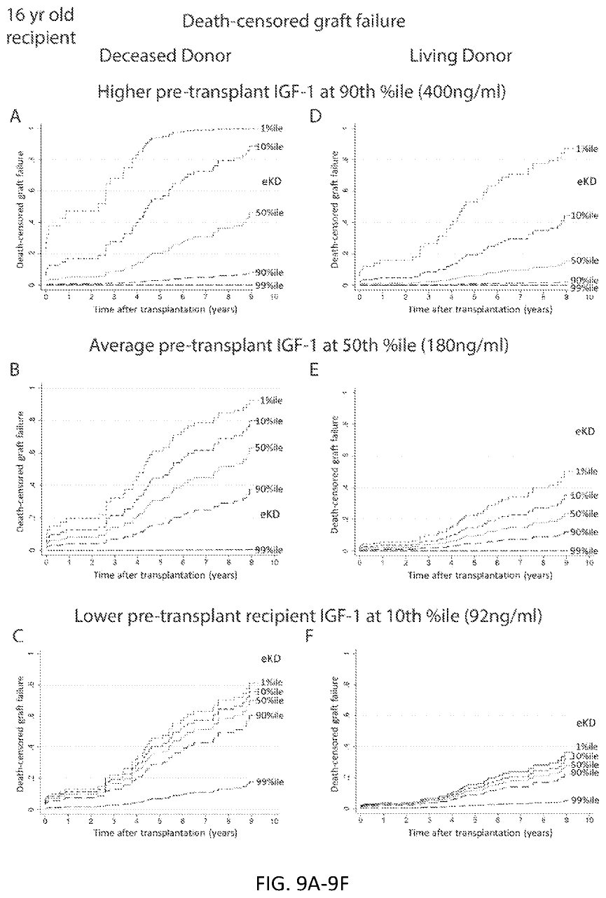

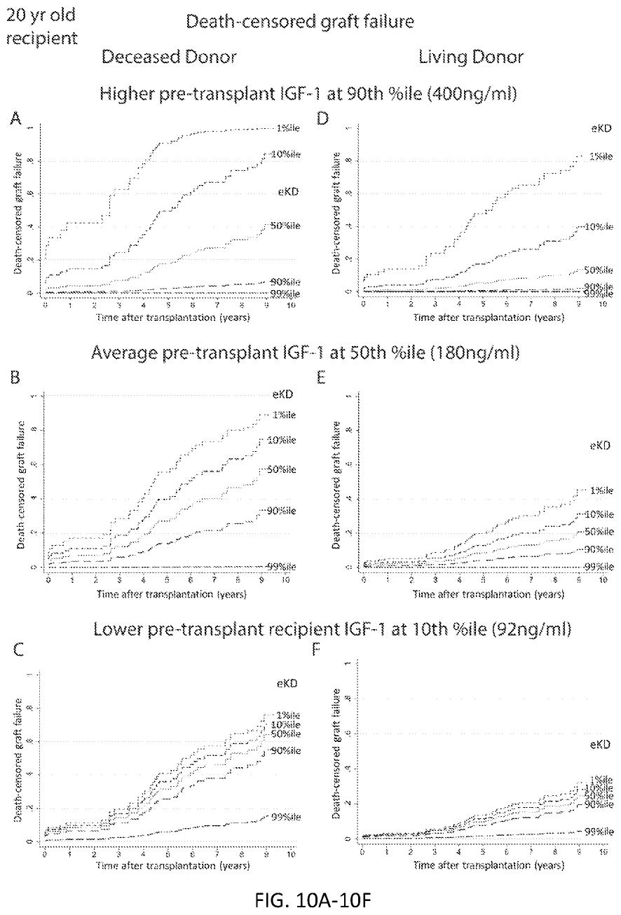

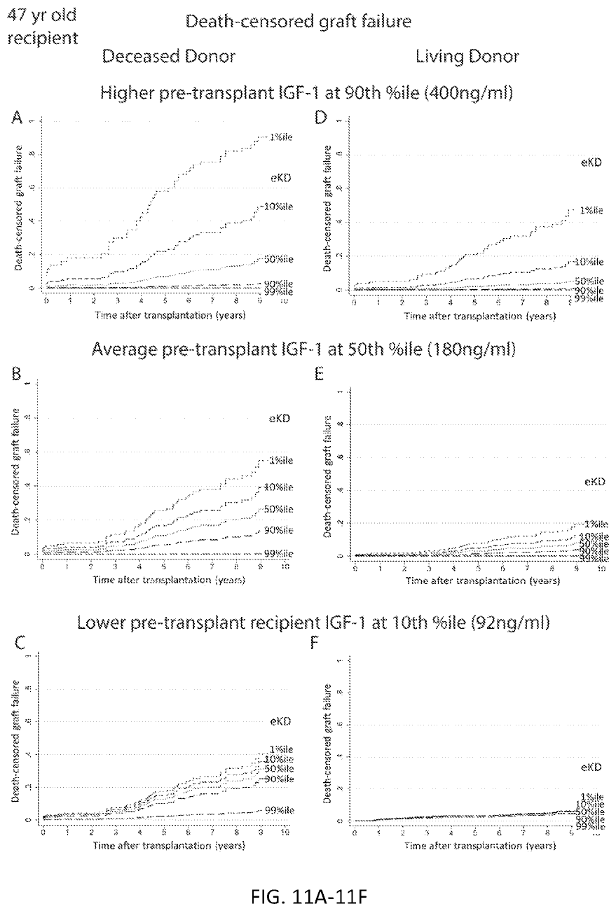

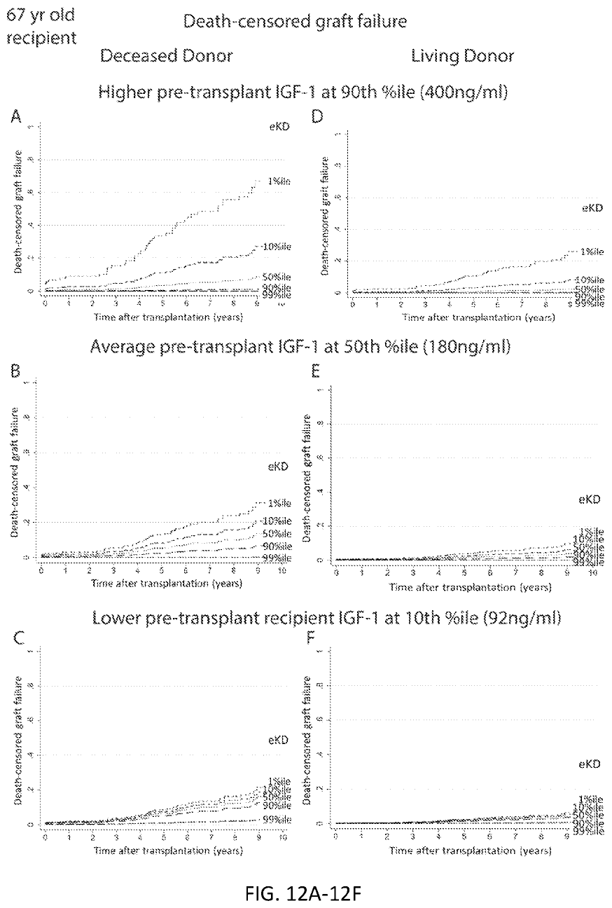

The effect of the interaction between IGF-1 level and eKD is illustrated in FIG. 4 (Panels A-F) for 40-year-old allograft recipients. Predicted death-censored allograft survival curves were constructed for high pre-transplant IGF-1 levels (90^{th} % ile=400 ng/ml), average IGF-1 levels (150^{th} % ile=180 ng/ml) and low IGF-1 levels (10^{th} % ile=92 ng/ml) according to whether the kidney dose delivered at transplantation (eKD) was very low (1^{th} % ile=0.5 kidneys), low (10^{th} % ile=0.74 kidneys), average (50^{th} % ile=1 kidney), high (90^{th} % ile=1.36 kidneys) or very high (99^{th} % ile=3.68 kidneys). FIG. 4 shows that at lower eKD, higher IGF-1 levels at the time of transplantation were associated with a marked reduction in allograft longevity. In contrast, at higher eKD, there was little to no association of higher IGF-1 with reduced allograft longevity. This reciprocal IGF-1/eKD effect was present for both deceased donor kidneys (Panel 4A-4C) and living donor kidneys (Panels 4D-4F), where a similar IGF-1/eKD effect was noted in the two cohorts. However, deceased donors had higher baseline graft loss. Similar allograft survival data in relation to pre-transplant IGF-1 levels and eKD were observed for 16, 20, 47, and 67-year-old recipients (shown in FIGS. 9, 10, 11, and 12). However, an additional superimposed effect of recipient age on allograft survival was present where the IGF-1/eKD effect was amplified in younger recipients and suppressed in older recipients. These data indicate that a reciprocal relationship exists between pre-transplant IGF-1 level and kidney dose. A higher pre-transplant IGF-1 and a smaller kidney dose were necessary to result in allograft failure. However, neither a high IGF-1 alone nor a smaller kidney dose alone was sufficient to drive allograft failure.

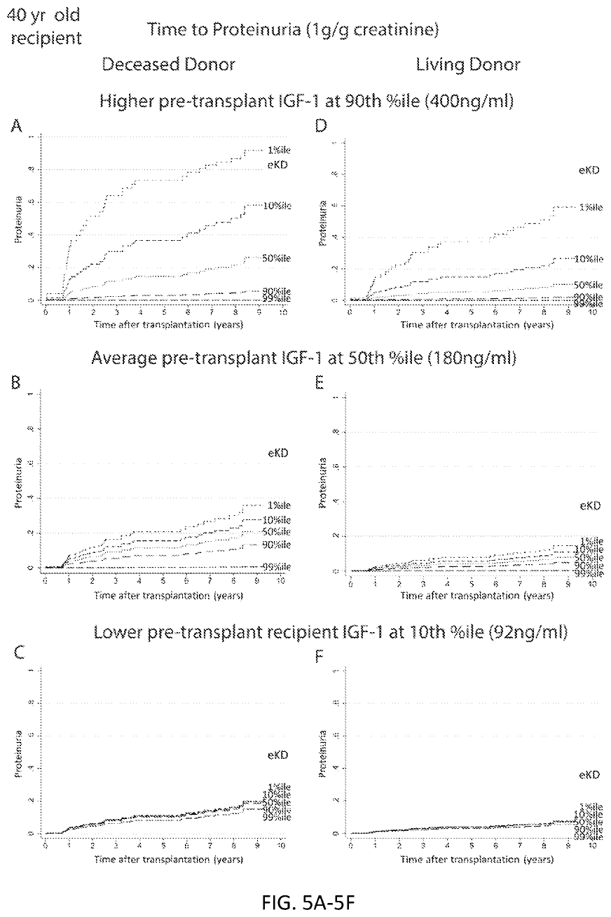

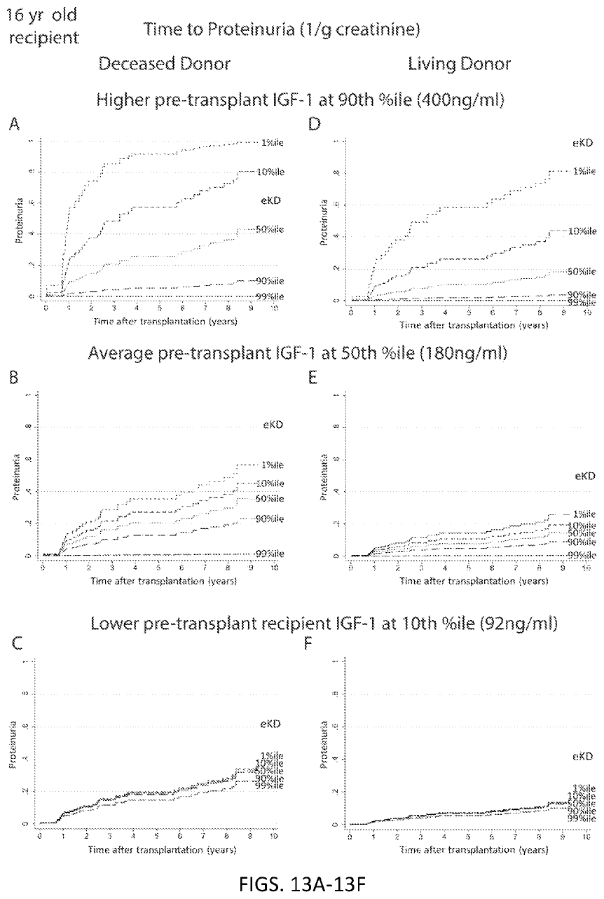

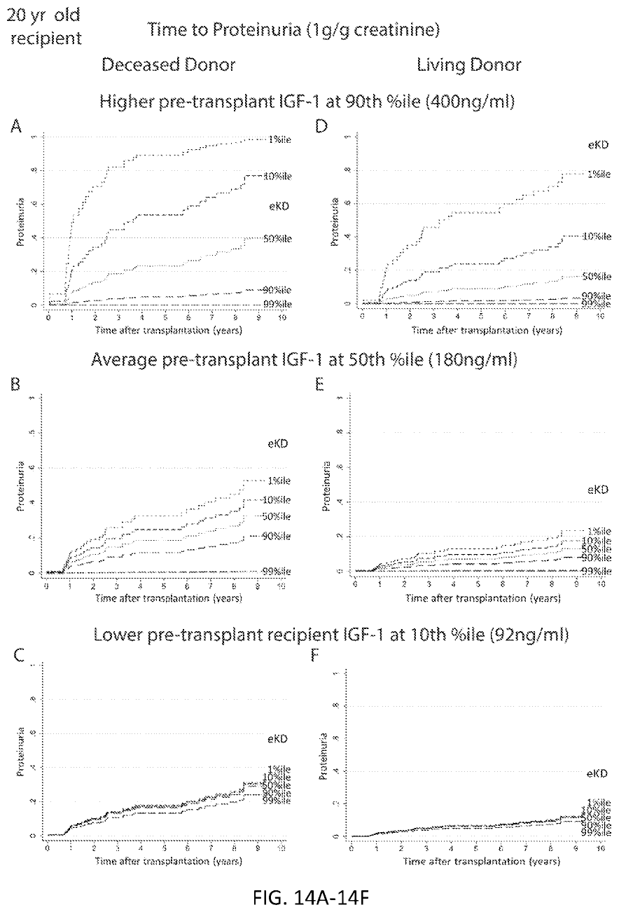

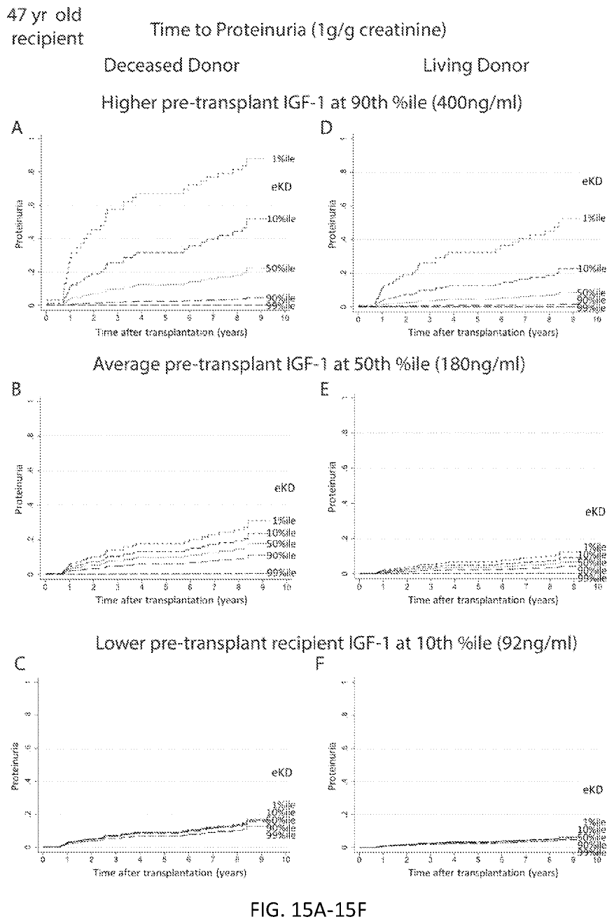

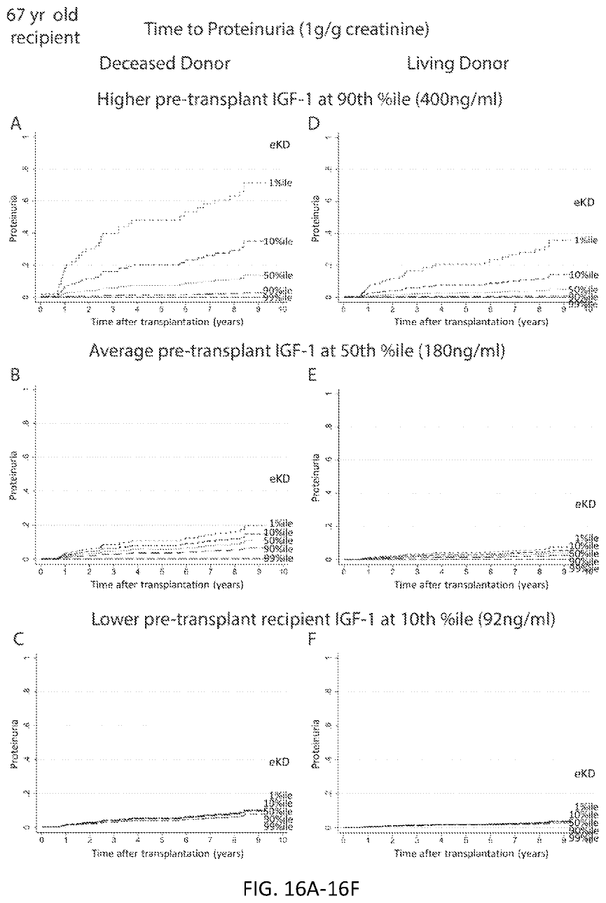

Effect of IGF-1 levels at the time of transplantation on time to post-transplant proteinuria at different levels of kidney dose. Proteinuria is a predictor of poor allograph outcomes. Table 5 shows a multivariable analysis demonstrating a significant relationship between blood IGF-1 at the time of transplantation and post-transplant proteinuria. As noted for the death-censored kidney allograft failure above, the interaction between IGF-1 and the eKD was significantly related to proteinuria (P=0.007). FIGS. 5 (Panels A-C) and (D-F) visually illustrate the effect of this interaction across cohorts of deceased and living donors, respectively, again demonstrating a significant association of higher IGF-1 levels with proteinuria at a low kidney dose that was not present at a high kidney dose. FIGS. 13, 14, 15, and 16 show similar proteinuria data for 16, 20, 47, and 67-year-old recipients, demonstrating a marked age effect on the IGF-1/eKD relationship to proteinuria.

TABLE 5
Multivariable cox model for the development
of post-transplant proteinuria

| | Hazard | Std. | | | 95% confidence | |
|---|---|---|---|---|---|---|
| | ratio | err. | z | P > z | interval | |
| IGF-1 level | 1.02 | 0.01 | 2.74 | 0.006 | 1.00 | 1.03 |
| eKD | 1.02 | 0.02 | 1.50 | 0.13 | 0.99 | 1.04 |
| Interaction term | 0.99 | 0.0001 | −2.70 | 0.007 | 0.99 | 0.99 |
| (IGF-1 * eKD) | | | | | | |

Other significantly related factors

| | | | | | | |
|---|---|---|---|---|---|---|
| Recipient age at TP | 0.97 | 0.01 | −1.76 | 0.08 | 0.95 | 1.00 |
| Recipient race | | | | | | |
| Black | 2.98 | 1.30 | 2.52 | 0.01 | 1.27 | 6.99 |
| Others | 0.87 | 0.80 | −0.16 | 0.88 | 0.14 | 5.28 |
| Male | 0.74 | 0.32 | −0.71 | 0.48 | 0.32 | 1.70 |
| Deceased donor | 2.81 | 1.20 | 2.43 | 0.02 | 1.22 | 6.50 |
| Donor age | 1.02 | 0.01 | 1.73 | 0.08 | 0.99 | 1.05 |
| Donor race | | | | | | |
| Black | 3.89 | 2.17 | 2.43 | 0.02 | 1.30 | 11.64 |
| Others | 4.12 | 2.62 | 2.22 | 0.03 | 1.18 | 14.38 |
| Previous TP | 0.08 | 0.54 | 0.15 | 0.88 | −0.97 | 1.13 |
| cPRA1 | −0.01 | 0.01 | −1.24 | 0.22 | −0.03 | 0.007 |
| cPRA2 | −0.01 | 0.01 | −0.92 | 0.36 | −0.03 | 0.01 |

Table legend:
BSA: Body Surface Area
D:R: Donor to Recipient
eKD: Effective Kidney Dose
IGF-1: Insulin-like growth factor 1
TP: Transplantation
cPRA: Calculated Panel Reactive Antibodies

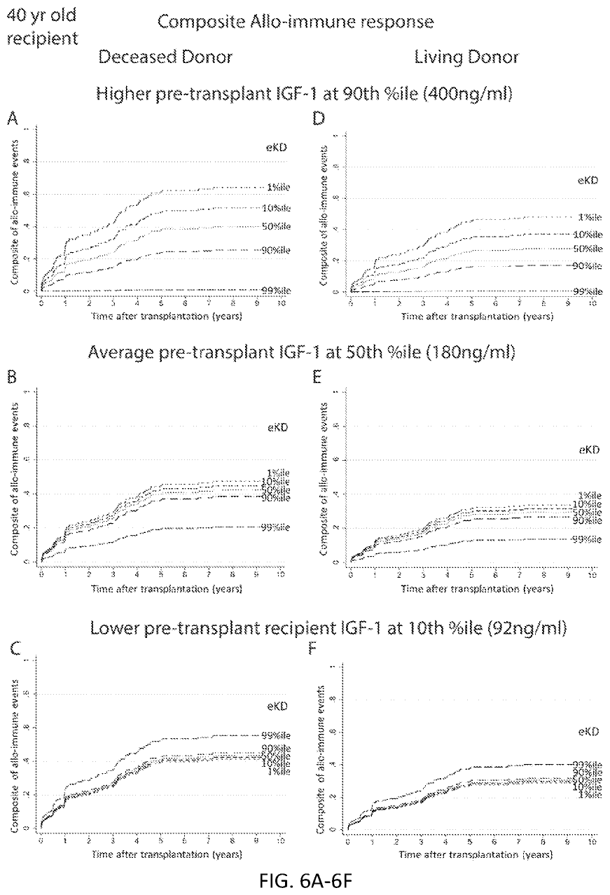

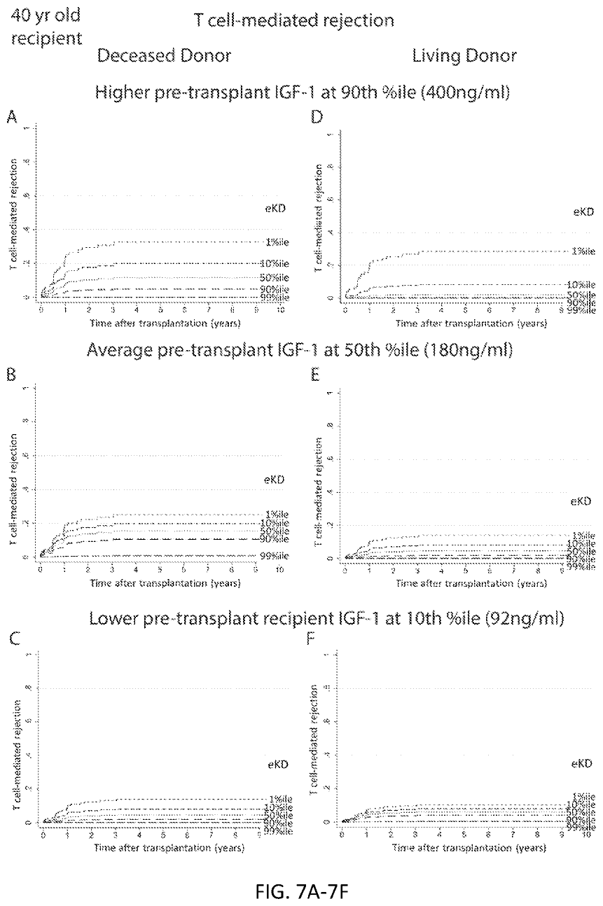

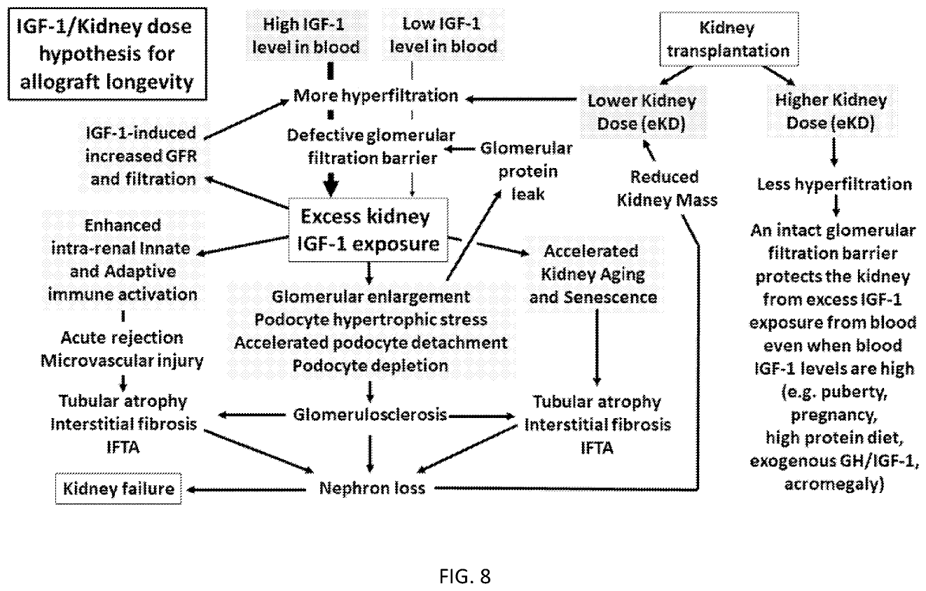

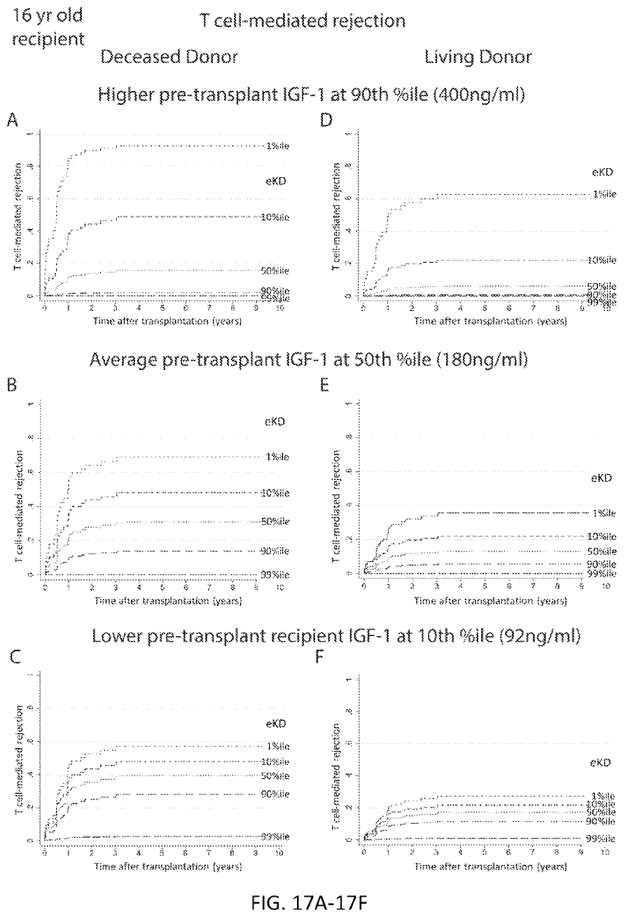

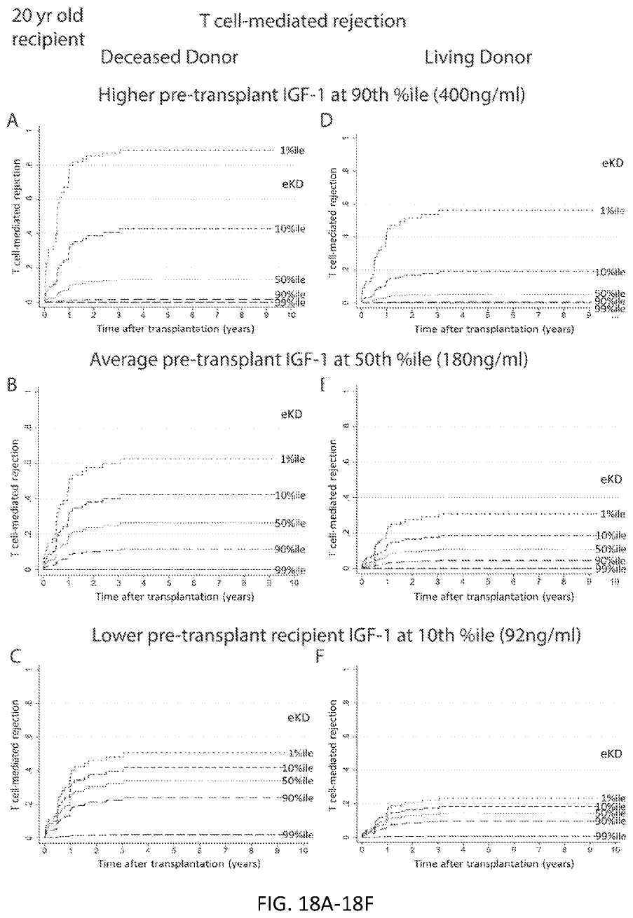

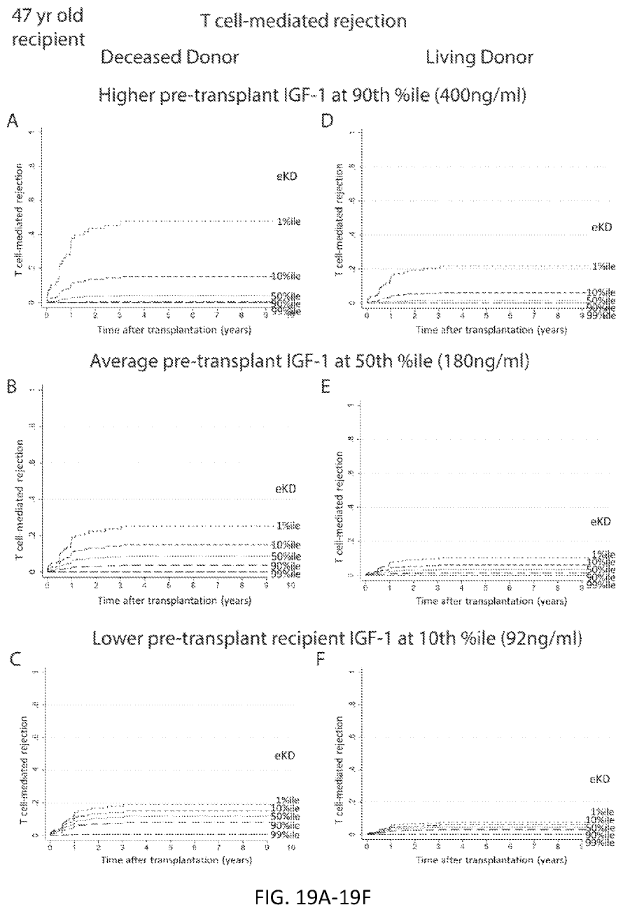

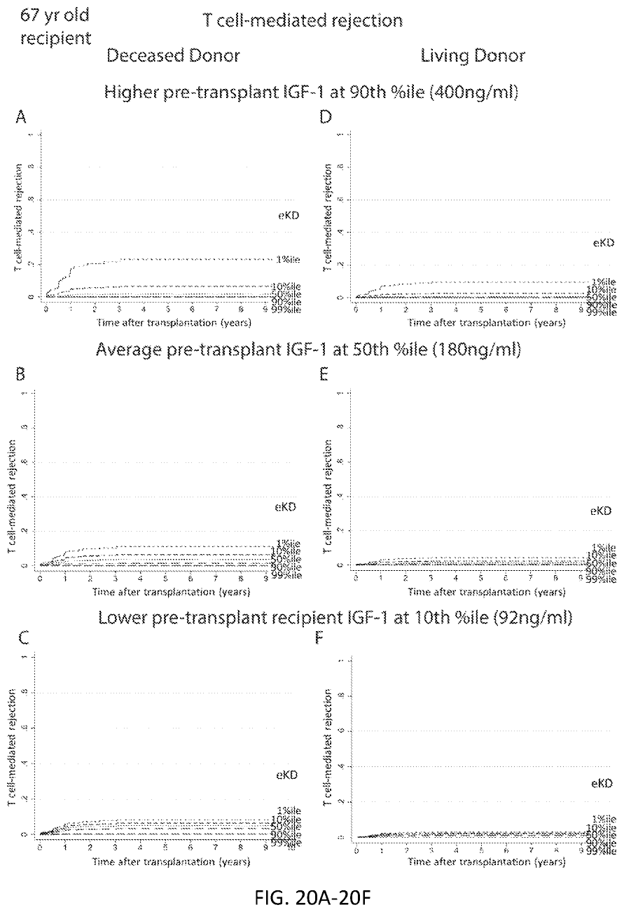

Effect of IGF-1 levels at the time of transplantation and kidney dose on the composite allo-immune response (time to biopsy-proven acute rejection and/or de novo donor-specific antibodies). Table 6 shows multivariable analysis demonstrating that the interaction between IGF-1 and the kidney dose (eKD) was significantly related to the composite allo-immune response (p=0.049). Thus, higher IGF-1 levels in the presence of lower kidney doses (predicted to cause more IGF-1 hyperfiltration) were associated with greater allo-immune response. The composite allo-immune response associated with the interaction between IGF-1 and eKD was driven mainly by biopsy-proven acute rejection (P=0.08) (Table 6), of which T-cell mediated rejection (as defined by Banff criteria) was statistically significant (P=0.047) (Table 7). FIGS. 6 and 7 show that higher blood IGF-1 levels were associated with alloimmunity when the eKD was lower and not observed when eKD was high, as noted above for allograft survival and proteinuria. FIGS. 17, 18, 19, and 20 show parallel biopsy-proven T cell-mediated rejection data for 16, 20, 47, and 67-year-old recipients, again demonstrating an age-modulating effect as noted for allograft survival and proteinuria.

TABLE 6
Multivariable Cox Model for composite alloimmune response.
Composite alloimmune response is the composite of
development of biopsy-proven rejection and/or donor
specific antibody, whichever was first.

| | Hazard | | | | 95% confidence | |
|---|---|---|---|---|---|---|
| | ratio | Std. err. | z | P > z | interval | |
| IGF-1 level | 1.00 | 0.003 | 1.48 | 0.14 | 0.99 | 1.01 |
| eKD | 1.01 | 0.01 | 1.20 | 0.23 | 0.99 | 1.03 |
| Interaction term | 0.99 | 0.00005 | −1.97 | 0.049 | 0.99 | 0.99 |
| (IGF-1 * eKD) | | | | | | |

Other significantly related factors

| | | | | | | |
|---|---|---|---|---|---|---|
| Recipient age at TP | 0.98 | 0.009 | −1.73 | 0.08 | 0.97 | 1.00 |
| Recipient race | | | | | | |
| Black | 1.05 | 0.31 | 0.18 | 0.86 | 0.59 | 1.88 |
| Others | 0.64 | 0.35 | −0.81 | 0.42 | 0.22 | 1.87 |
| Male | 0.69 | 0.19 | −1.37 | 0.17 | 0.41 | 1.17 |
| Deceased donor | 1.57 | 0.43 | 1.65 | 0.1 | 0.92 | 2.68 |
| Donor age | 0.99 | 0.009 | −0.69 | 0.49 | 0.97 | 1.01 |
| Donor race | | | | | | |
| Black | 3.47 | 1.40 | 3.08 | 0.002 | 1.57 | 7.66 |
| Others | 3.29 | 1.37 | 2.86 | 0.004 | 1.45 | 7.44 |
| Previous TP | 0.62 | 0.23 | −1.27 | 0.20 | 0.29 | 1.30 |
| cPRA1 | 1.00 | 0.006 | 0.34 | 0.74 | 0.99 | 1.01 |
| cPRA2 | 1.01 | 0.007 | 1.65 | 0.10 | 0.99 | 1.02 |

Table legend:
BSA: Body Surface Area
D:R: Donor to Recipient

TABLE 7
Multivariable Cox Model for post-transplant
T-Cell mediated rejection.

| | | | | | 95% | |
|---|---|---|---|---|---|---|
| | Hazard | Std. | | | confidence | |
| | ratio | err. | z | P > z | interval | |
| IGF-1 level | 1.01 | 0.008 | 1.39 | 0.16 | 0.99 | 1.03 |
| eKD | 1.005 | 0.019 | 0.28 | 0.78 | 0.97 | 1.04 |
| Interaction term | 0.99 | 0.0002 | −1.96 | 0.05 | 0.99 | 1.00 |
| (IGF-1 * eKD) | | | | | | |

Other significantly related factors

| | | | | | | |
|---|---|---|---|---|---|---|
| Recipient age at TP | 0.96 | 0.02 | −2.47 | 0.01 | 0.92 | 0.99 |
| Recipient race | | | | | | |
| Black | 0.46 | 0.27 | −1.34 | 0.18 | 0.15 | 1.44 |
| Others | 1.81 | 1.39 | 0.78 | 0.43 | 0.41 | 8.15 |
| Male | 0.38 | 0.18 | −2.00 | 0.046 | 0.15 | 0.98 |
| Deceased donor | 2.65 | 1.28 | 2.02 | 0.04 | 1.03 | 6.85 |
| Donor age | 0.98 | 0.02 | −1.42 | 0.15 | 0.95 | 1.00 |
| Donor race | | | | | | |
| Black | 11.4 | 8.03 | 3.46 | 0.001 | 2.88 | 45.33 |
| Others | 9.80 | 4.98 | 4.49 | <0.0001 | 3.62 | 26.5 |
| Previous TP | 1.27 | 0.60 | 0.51 | 0.61 | 0.51 | 3.18 |
| cPRA1 | 0.98 | 0.01 | −1.47 | 0.14 | 0.96 | 1.00 |
| cPRA2 | 1.02 | 0.01 | 1.29 | 0.20 | 0.99 | 1.04 |

Table Legend:
BSA: Body Surface Area
D:R: Donor to Recipient
eKD: Effective Kidney Dose
IGF-1: Insulin-like growth factor 1
TP: Transplantation
cPRA: Calculated Panel Reactive Antibodies

Tables 4, 5, 6, and 7 show that race of the receipt and/or donor being black were associated with worse outcomes, as is known. No specific interaction of race with IGF-1 was observed. Recipient or donor sex was not related to graft failure or proteinuria.

In parallel analyses, pre-transplant IGF-2 level was not significantly associated with kidney dose, time-to-death censored kidney allograft failure, proteinuria, or allo-immune response (data not shown).

### Transcriptomic Study

This analysis focused on IGF-1, IGF-2, IGF-1 receptor (IGF-1R), Growth Hormone receptor (GHR), and the Insulin Receptor (INSR) and Insulin-like Growth Factor Binding Proteins (IGFBP1-IGFBP6) that bind IGF-1 with 10-100-fold higher affinity than does the IGF-1R. First-year post-transplant protocol biopsies with no detectable histologic abnormality were compared to matched samples from healthy live donors obtained at time of transplantation.

### GH, IGF-1, IGF-2, IGF-1R and INSR Transcript Expression in Normal Human Kidneys

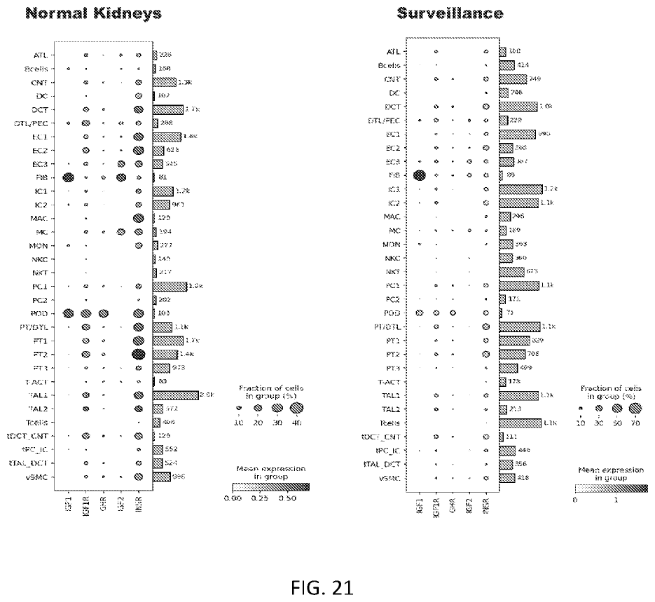

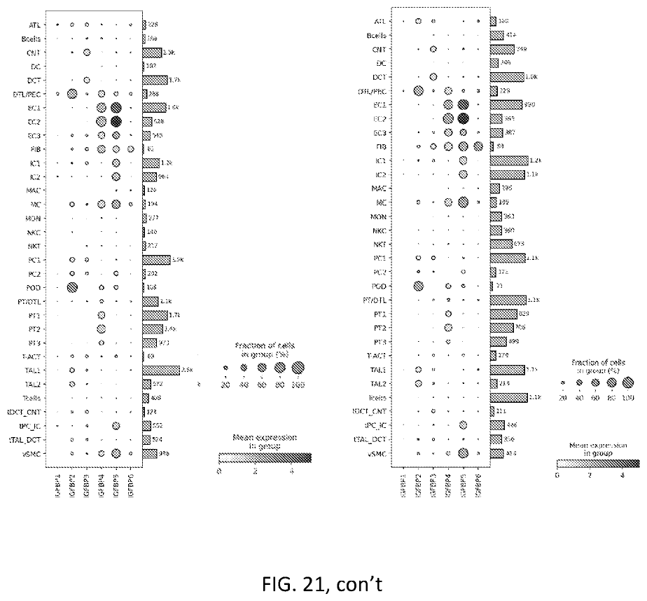

FIG. 21 shows that IGF-1 transcript was highly expressed in podocytes (Pod) and fibroblasts (FIB) but was low or undetectable in most other kidney cell types. IGF-1R was ubiquitously expressed with higher level expression in podocytes and subclusters of proximal tubular cells (PT), thick ascending limb cells (TAL) and distal tubular/collecting duct cells (DTL-CNT). Growth hormone receptor (GHR) was predominantly expressed by podocytes. INSR expression tended to parallel IGF-1R transcript expression, consistent with INSR and IGF-1R being gene reduplicates, and that IGF-1R and INSR form heterodimeric receptors that bind and are activated by IGF-1. IGF2 was expressed by mesangial cells (MC), fibroblasts (FIB) and peritubular capillaries (EC3). GH transcript was not detected in kidney cells (not shown).

### IGFBP Expression in Healthy Normal Human Kidneys

FIG. 21 further shows that IGFBP 1-6 expression in normal kidney was quite cell specific.

### Changes in IGF-1 Pathway Transcript in Allografts

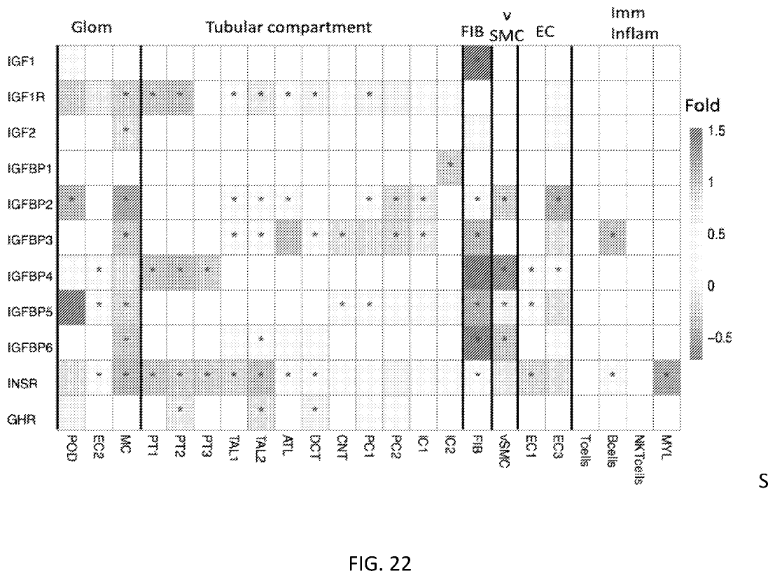

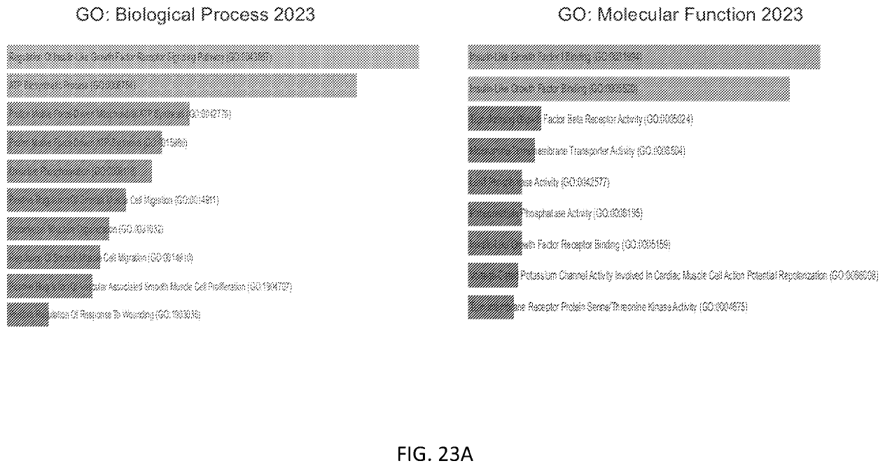

FIG. 21 further shows IGF-1 pathway transcript expression in allografts. FIG. 22 shows a heat map quantitating differences in transcript expression levels for different cell types and whether these differences achieved statistical significance. IGF-1 and GHR transcript expression was decreased in allograft podocytes and undetectable in most other kidney cells. IGF-1R transcript tended to be decreased in all allograft cells. In podocytes the decrease in allograft IGF-1, IGF-1R and GHR transcript expression did not achieve statistical significance. IGF-2 transcript was significantly reduced in mesangial cells.

### Fibroblasts

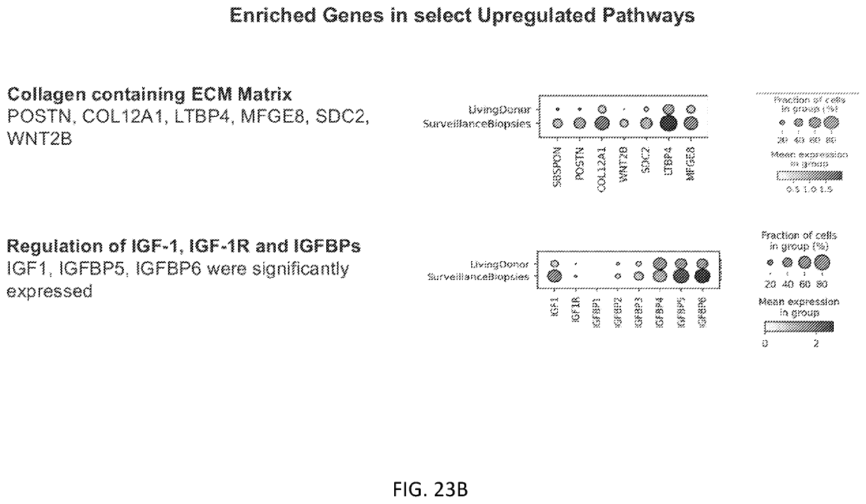

FIG. 22 shows that the largest changes in IGF-1 pathway expression occurred in fibroblasts. IGF-1 and IGFBP3, IGFBP4 and IGFBP5 expression were significantly increased in allograft fibroblasts. This identifies fibroblasts as a major potential source of intrinsic IGF-1 production, even in stable normally functioning allografts without histologic abnormality. Additional studies were therefore performed to evaluate the fibroblast transcriptome. Gene Ontology tools including Biological Processes and Molecular Functions identified IGF-1-related pathways as upregulated (FIG. 23A). Genes identified as significantly upregulated included those associated with extracellular matrix (ECM) including SBSPON, POSTN, COL12A1, WNT2B, SDC2, LTBP4 and MFGE8 (FIG. 23B).

### Discussion

The data presented herein indicate that allograft longevity is related to recipient IGF-1 level at time of kidney transplantation. Higher pre-transplant IGF-1 level was related to both shorter allograft survival and the development of proteinuria, a strong predictor of long-term allograft outcome. Moreover, it was observed that allograft survival is inversely and closely related to the age-predicted IGF-1 level at time of transplantation.

The relationship of IGF-1 to shorter allograft survival depended in part on the kidney dose delivered at transplantation (eKD). Kidney dose and recipient BMI in turn, determines relative kidney blood flow and degree of hyperfiltration. At higher circulating IGF-1 levels, a smaller kidney dose was associated with more graft loss and increased proteinuria. Conversely, at low circulating IGF-1 levels, the donor kidney mass was much less consequential for graft outcome. This indicates that relative kidney blood flow x IGF-1 blood concentration reflects kidney IGF-1 exposure that impacts allograft longevity. It also indicates that allograft IGF-1 exposure is at least in part extrinsic, being derived from the blood compartment where IGF-1 is stored at high concentration.

Normal kidney podocytes express high level IGF-1, GHR and IGF-1R transcripts, indicating that the kidney has its own intrinsic IGF-1 production and regulatory mechanism under control of pituitary-derived GH that can cross the filtration barrier to access podocyte-expressed GHR and thereby both promote IGF-1 production to regulate growth. GH-induced podocyte depletion may plays a role in diabetic glomerulosclerosis. The transcriptomic data provided herein support this hypothesis, and further implicate IGF-1 production by podocytes and a negative feedback regulation of IGF-1 production through the IGF-1R. Under hyperfiltration conditions podocytes would be exposed to both increased GH (tending to up-regulate IGF-1 production and growth) and IGF-1 (that would tend to down-regulate podocyte IGF-1 production while at the same time activating the mTORC1 pathway to promote growth). Thus, hyperfiltration sets into place counteracting forces on podocytes driving hypertrophic stress and accelerated detachment. Model systems show that GH itself appears to amplify IGF-1 effects on the kidney. For example, in transgenic mice that over-express IGF-1, enlarged glomeruli were observed but not FSGS. In contrast, mice made transgenic to over-express GH with similarly high circulating IGF-1 levels, developed both glomerular enlargement and FSGS (Blutke A, Schneider M R, Wolf E, Wanke R. Growth hormone (GH)-transgenic insulin-like growth factor 1 (IGF 1)-deficient mice allow dissociation of excess GH and IGF 1 effects on glomerular and tubular growth. Physiol Rep. 2016; 4(5):e12709). Notably, GH levels are reported to be increased in kidney failure, thus at transplantation allograft podocytes would be exposed to particularly high GH levels (Mak R H, Cheung W W, Roberts Jr C T. Growth Horm IGF Res. 2008; 18(1):17-25). Thus, although this report focuses on IGF-1 at the time of transplantation, blood IGF-1 level may also serve as a surrogate for circulating GH levels or a combination of GH and IGF-1 effects.

Fibroblasts in normal kidney express high level IGF-1 transcript which was further amplified in stable allografts. Thus, fibroblasts are a potential source of intrinsic kidney IGF-1 in allografts. For example, stable allograft fibroblasts expressed POSTN, and other transcripts associated with increased extracellular matrix (ECM) deposition (SBSPON, COL12A1, WNT2B, SDC2, LTBP4 and MFGE8). The IGF-1*eKD interaction was also associated with an elevation in composite alloimmune responses against the kidney allograft (predominantly driven by biopsy-proven T-cell-mediated rejections of grade 1A or higher).

The population study showed that the peak rate of graft loss coincides with the highest circulating levels of IGF-1, which occur during puberty. Non-compliance in teen-age years has previously been suggested as a cause for increased graft loss. The data presented herein suggests that higher IGF-1 levels in this age range is an additional factor capable of driving graft failure.

Free IGF-1 (7.5 kDa) makes up only about 1% of total IGF-1 in blood. The remaining IGF-1 (and IGF-2) circulates bound to IGF-binding proteins (IGFBP1-5), a family of gene reduplicated proteins that bind IGF-1 with 10-100-fold higher affinity than does the IGF-1R and thereby regulates local IGF-1 availability. IGFBPs release IGF-1 in response to proteolytic and stereochemical modulations induced by binding to matrix and other molecules. IGF-IGFBP bi-molecular complexes have molecular weights ranging from about 25-45 kDa. The major IGF-BPs in blood are IGFBP3 and IGFBP5 which also bind to the Acid Labile Subunit (ALS) in blood to form trimolecular 150 kDa complexes that have a longer half-life. IGF-1 (and IGF-2) are thus stored in blood at high concentration in dynamic equilibrium with their binding proteins. Both free IGF-1 and the IGF-IGFBP bi-molecular complexes would be expected to cross the glomerular filtration barrier to gain access to the urinary space, especially under hyperfiltration conditions. Increased renal blood flow in the allograft would also increase IGF access to the kidney interstitial space and basolateral surface of tubular cells via peritubular capillaries. IGF-2 was present in pre-transplant serum at 3-5 times the concentration of IGF-1. IGF-2 binds to the same IGFBPs and to ALS that serve as carriers of both IGF-1 and IGF-2, and IGF-1 functions by activating the same widely expressed IGF-1R in tissues. Therefore, pre-transplant IGF-2 levels might also have been expected to be related to allograft longevity. However, no significant relationship was found herein between pre-transplant IGF-2 levels and allograft longevity, proteinuria, or allo-immune events. This suggests that IGF-2 may not be hyperfiltered into the kidney to the same extent as IGF-1. Consistent with this possibility, previous studies in a rat uni-Nx model demonstrated that IGF-1 was preferentially hyperfiltered to appear in urine compared to IGF-2 (Naik A S, et al. Critical timing of ACEi initiation prevents compensatory glomerular hypertrophy in the remaining single kidney. Sci Rep. 2021; 11(1):19605. doi: 10.1038/s41598-021-99124-z).

Increasing transplanted kidney dose may minimize the impact of high IGF-1 levels on allograft outcome (e.g. during adolescence). Reducing early allograft exposure to GH/IGF-1 may improve long-term allograft survival. ACE inhibition, by reducing hyperfiltration and by regulating intrarenal IGF-1 transcription and thereby limiting graft exposure to GH/IGF-1, would be expected to be protective. However, several ACE inhibitor trials in allografts have shown little clinical benefit, although recent evidence does suggest that early versus later drug initiation may improve surrogates of tissue injury and long-term function (Cockfield S M, et al. Am J Transplant. Published online 2018; Shiffermiller J F, et al. J Hosp Med. 2018; 13(10):661-667. doi: 10.12788/jhm.3036).

The data presented herein raises the possibility that the lack of an ACE inhibition protective effect could occur because drug was not started until well after transplantation due to concern for potential side-effects (hyperkalemia, reduced renal perfusion, hypotension and anemia). The data herein suggests that the major growth events that ultimately determine allograft destiny occur very early after transplantation. Alternative approaches to extend allograft survival include specific IGF-1R blockade (currently in clinical use to treat auto-immune thyroid eye disease (Douglas R S, et al. Ophthalmology. 2022; 129(4):438-449.) and/or targeting GH by GHRH inhibition using octreotide or its analogues.

### Methods

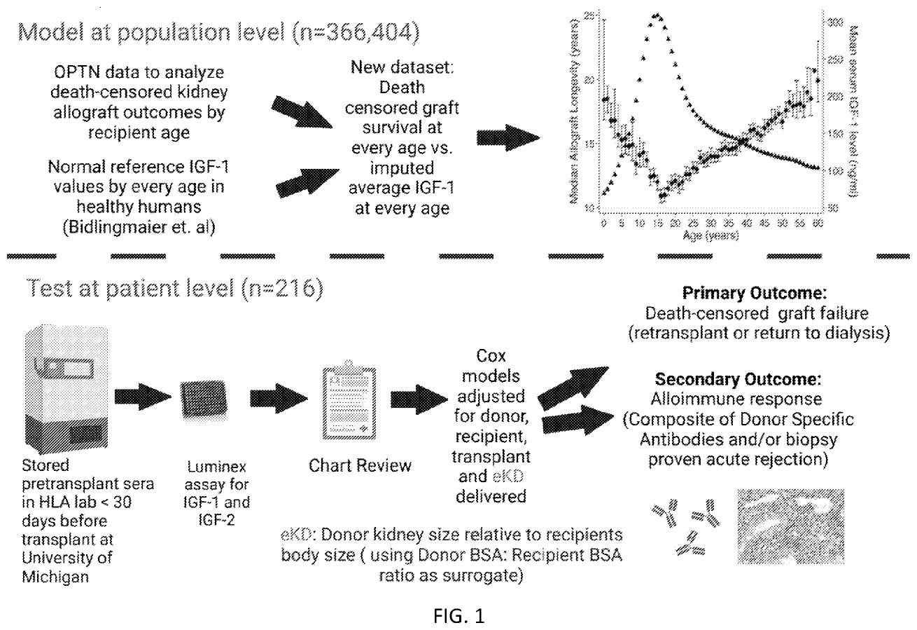

FIG. 1 illustrates exemplary approaches taken herein.

### Clinical Level Study

Power analysis: A sample size estimation of 211 patients was calculated using a hazard ratio (HR) of 2.0, an event probability of 0.31, and a power of 0.8 and was based on an interim analysis of the initial 106 recipients. The HRs from previously reported uni-Nx animal data ranged from 4-8-fold.

Data set: Stored serum samples obtained before transplantation were used for 216 recipients who subsequently underwent kidney transplantation.

Luminex assay for IGF-1 and IGF-2: Human IGF-1 and IGF-2 were measured in human serum by MILLIPLEX MAP human IGF-I and IGF II magnetic bead panels (EMD Millipore, Billerica, MA).

Effective Kidney Dose (eKD): eKD was calculated for each recipient as the % replacement of the normal kidney dose and given by ((donor BSA/recipient BSA)/(2))*100. Thus, an eKD at the 50^{th} % ile=1 kidney equivalent for that recipient.

Outcomes of Interest: The primary outcome of interest for which the study was powered was the time-to-death-censored kidney allograft failure from any cause. The secondary outcomes of interest were time to development of proteinuria (defined as ≥1 g/g creatinine) and time to either biopsy-proven acute rejection (BPAR) or the detection of Class 1, Class 2 donor-specific antibodies (DSA), or both. Subgroup analysis of individual components of the alloimmune response included biopsy-proven acute rejection (BPAR), de novo DSA, Class 1, or Class 2 DSA, and cellular, mixed, or antibody-mediated rejection identified by protocol biopsies at 3, 6, and 12 months and indication biopsies at any time after transplantation (See Supplemental Methods). Delayed Graft Function (DGF) was defined as the need for dialysis in the first 7 days post kidney transplantation.

Survival analysis and Cox and Logistic regression: Time-to-event analysis of the primary and secondary hypothesis (and subgroup analysis) was performed using Cox regression analysis to assess the effect of pre-transplant IGF-1 level on outcomes. An interaction of IGF-1 at the time of transplantation with the eKD delivered was included due to the underlying hypothesis that the amount of IGF-1 delivered perioperatively to the allograft would be a function of the degree of glomerular hyperfiltration and the IGF-1 blood concentration at the time of transplantation. Adjustments of other covariates known to drive long-term outcomes were included a priori (see Table 1 legend). A logistic regression model was used to test whether the interaction of pretransplant IGF-1 levels and eKD was associated with delayed graft function.

Statistical Analysis: All continuous variables were reported as mean values±standard deviation. All categorical variables were reported as proportions. Correlation between two continuous variables was performed by Pearson correlation. After the interim analysis, Stata command power Cox was used for sample size estimation. The Cox proportional hazards assumption was verified based on the Schoenfeld residuals implemented in the Stata command estat phtest. All analysis was performed using Stata/MP 17.0 (College Station, Texas).

### Population-Level Study.

Median time to death censored graft failure: The Organ Procurement and Transplantation Network (OPTN) database was used. After exclusion criteria were applied, 366,404 observations were available for further analysis. Time to median death-censored graft failure (DCGF) (“allograft longevity”) for every year of recipient age between 0-60-years was obtained using the Kaplan-Meier estimator censored for death, re-transplantation, loss to follow-up, or end of the follow-up period.

IGF-1 for each age group: Population-level data for the distribution of average serum IGF-1 levels by age and gender across 0-60 years for a normal population was obtained from Bidlingmaier et al. (n=15,014) (Bidlingmaier M, Friedrich N, Emeny R T, et al. J Clin Endocrinol Metab. 2014; 99(5):1712-1721.

Transcriptomic analysis using the Human Kidney Transplant Transcriptomic Atlas (HKTTA): The HKTTA study collected longitudinal biosamples in an unbiased fashion from patients consenting an extra tissue core during their standard of care and indication surveillance kidney biopsies as previously described (Menon R, et al. Kidney Int. 2022; 101 (4): 779-792. doi: 10.1016/j.kint.2021.11.031).

Rationale for eKD estimation: Allograft IGF-1 exposure from blood is a function of the IGF-1 concentration in the recipient blood and the degree of allograft hyperfiltration. The latter arises from two factors: (i) the allograft being a single kidney that replaces the normal two-kidney complement, and (ii) the donor-to-recipient body size ratio (effective kidney dose or eKD). We therefore calculated an eKD for each recipient to denote allograft kidney dose as a percentage of the ideal two-kidney complement. For example, if a donor and recipient have the same body surface area, then the eKD for a single kidney for that recipient is 50%. Thus, allograft IGF-1 exposure at transplantation is denoted by the interaction term of IGF-1 level x eKD.

### Transcriptomic Information:

Single cell processing: The generation of single cell preparations was accomplished by enzymatic (Liberase TL; Sigma Aldrich) and mechanical dissociation for 12 minutes at 37° C. Cells are filtered through a 30 μm strainer and counted, and up to 10,000 viable cells are submitted for droplet-based high-throughput scRNA-seq on the 10× Genomics Chromium platform. After droplet encapsulation, the 10× Genomics approach allows individual cell RNA molecular barcoding, and reverse transcription. Subsequently, cDNA libraries are generated and sequenced on the NovaSeq 6000 platform (Illumina) as asymmetric paired-end (26×151) runs and generating >200 million raw sequence reads per sample. The sequencing data are preprocessed using the 10× Genomics software Cell Ranger. Downstream analysis is performed with the Seurat R package version 3. A combined analysis of the single cell data sets generated from the different sample sources (living donor and surveillance biopsies) using Seurat version 3 includes the following steps: filtering of cells with <500 genes or >50% mitochondrial content default normalization, scaling based on sample mRNA count and mitochondrial RNA content, dimensionality reduction principal component analysis and uniform manifold approximation and projection, sample integration using the Harmony algorithm, standard unsupervised clustering, and the discovery of differentially expressed cell 5 type-specific markers.

1. A method of extending lifespan of a transplant, the method comprising providing an inhibitor of insulin-like growth factor 1 receptor (IGF-1R) signaling and/or an inhibitor of growth hormone receptor (GHR) signaling to a subject before, during, and/or after a transplant in the subject.

2. The method of claim 1, wherein the transplant is a kidney transplant, a lung transplant, or a heart transplant.

3. The method of claim 2, wherein the transplant is a kidney transplant.

4. The method of any one of claims 1-3, wherein the inhibitor of IGF-1R signaling and/or the inhibitor of GHR signaling is provided to the subject within 30 days of the transplant.

5. The method of claim 4, wherein the inhibitor of IGF-1R signaling and/or the inhibitor of GHR signaling is provided to the subject within 3 weeks of the transplant.

6. The method of claim 5, wherein the inhibitor of IGF-1R signaling and/or the inhibitor of GHR signaling is provided to the subject within 2 weeks of the transplant.

7. The method of claim 6, wherein the inhibitor of IGF-1R signaling and/or the inhibitor of GHR signaling is provided to the subject within 10 days of the transplant.

8. The method of claim 7, wherein the inhibitor of IGF-1R signaling and/or the inhibitor of GHR signaling is provided to the subject within 7 days of the transplant.

9. The method of claim 8, wherein the inhibitor of IGF-1R signaling and/or the inhibitor of GHR signaling is provided to the subject within 5 days of the transplant.

10. The method of claim 9, wherein the inhibitor of IGF-1R signaling and/or the inhibitor of GHR signaling is provided to the subject within 3 days of the transplant.

11. The method of any one of claims 1-10, comprising providing an inhibitor of IGF-1R signaling to the subject.

12. The method of claim 11, wherein the inhibitor of IGF-1R signaling comprises a small molecule inhibitor.

13. The method of claim 12, wherein the inhibitor of IGF-1R signaling comprises a small molecule inhibitor selected from Linsitinib, NVP-AEW541, GSK1904529A, NVP-ADW742, BMS-536924, Ceritinib, AG-1024, GSK1838705A, BMS-754807, PQ 401, Ginsenoside Rg5, Ceritinib dihydrochloride, XL228, Brigatinib, Picropodophyllin, AZD3463, Insulin Degludec, S961, and Nordihydroguaiaretic acid (NGDA).

14. The method of claim 13, wherein the inhibitor of IGF-1R signaling comprises an antibody.

15. The method of claim 14, wherein the antibody is selected from Teprotumumab, Dalotuzumab, Figitumumab, Ganitumab, Robatumumab, and Cixutumumab.

16. The method of any one of claims 1-10, comprising providing an inhibitor of GHR signaling to the subject.

17. The method of claim 16, wherein the inhibitor of GHR signaling is a protein or an antibody.

18. The method of claim 17, wherein the inhibitor of GHR signaling is Pegvisomant.

19. The method of any one of claims 1-18, wherein the subject is an adult subject.

20. The method of any one of claims 1-18, wherein the subject is a peripubertal subject.

21. The method of any one of claims 1-18, wherein the subject is a prepubertal subject.

22. The method of any one of claims 1-21, wherein the transplant is a kidney transplant, and wherein levels of IGF-1 in a sample obtained from the subject prior to kidney transplant are above a threshold value for IGF-1 and/or wherein an effective kidney dose (eKD) of the kidney transplant is below a threshold value for eKD.

23. A method of determining potential lifespan of a kidney transplant in a subject, comprising:
a) determining the effective kidney dose (eKD) of the kidney transplant;
b) determining levels of IGF-1 in a sample obtained from the subject, and
c) determining potential lifespan of the kidney transplant in the subject based at least in part upon the eKD and the levels of IGF-1 in the sample obtained from the subject.

24. The method of claim 23, further comprising determining one or more factors selected from age of the organ donor, status of the organ donor, age of the subject, and race of subject, wherein the one or more factors are additionally used in the determination of potential lifespan of the kidney transplant.

25. The method of claim 23 or claim 24, comprising:
a) determining a poor lifespan of the kidney transplant in the subject when levels of IGF-1 in the sample are equal to or above a threshold value and the eKD is below a threshold value, or
b) determining an acceptable lifespan of the kidney transplant when the eKD is equal to or above a threshold value; or
c) determining an acceptable lifespan of the kidney transplant when the levels of IGF-1 in the sample are below a threshold value.

26. The method of any one of claims 23-25, further comprising providing an inhibitor of insulin-like growth factor 1 receptor (IGF-1R) signaling and/or an inhibitor of growth hormone receptor (GHR) signaling to the subject when a poor lifespan of the kidney transplant is determined.

27. The method of claim 26, wherein the inhibitor of IGF-1R signaling and/or the inhibitor of GHR signaling is provided to the subject within 30 days of the kidney transplant.

28. The method of claim 27, wherein the inhibitor of IGF-1R signaling and/or the inhibitor of GHR signaling is provided to the subject within 3 weeks of the kidney transplant.

29. The method of claim 28, wherein the inhibitor of IGF-1R signaling and/or the inhibitor of GHR signaling is provided to the subject within 2 weeks of the kidney transplant.

30. The method of claim 29, wherein the inhibitor of IGF-1R signaling and/or the inhibitor of GHR signaling is provided to the subject within 10 days of the kidney transplant.

31. The method of claim 30, wherein the inhibitor of IGF-1R signaling and/or the inhibitor of GHR signaling is provided to the subject within 7 days of the kidney transplant.

32. The method of claim 31, wherein the inhibitor of IGF-1R signaling and/or the inhibitor of GHR signaling is provided to the subject within 5 days of the kidney transplant.

33. The method of claim 32, wherein the inhibitor of IGF-1R signaling and/or the inhibitor of GHR signaling is provided to the subject within 3 days of the kidney transplant.

34. The method of any one of claims 26-33, comprising providing an inhibitor of IGF-1R signaling to the subject.

35. The method of claim 34, wherein the inhibitor of IGF-1R signaling comprises a small molecule inhibitor.

36. The method of claim 35, wherein the inhibitor of IGF-1R signaling comprises a small molecule inhibitor selected from Linsitinib, NVP-AEW541, GSK1904529A, NVP-ADW742, BMS-536924, Ceritinib, AG-1024, GSK1838705A, BMS-754807, PQ 401, Ginsenoside Rg5, Ceritinib dihydrochloride, XL228, Brigatinib, Picropodophyllin, AZD3463, Insulin Degludec, S961, and Nordihydroguaiaretic acid (NGDA).

37. The method of claim 36, wherein the inhibitor of IGF-1R signaling comprises an antibody.

38. The method of claim 37, wherein the antibody is selected from Teprotumumab, Dalotuzumab, Figitumumab, Ganitumab, Robatumumab, and Cixutumumab.

39. The method of any one of claims 26-33, comprising providing an inhibitor of GHR signaling to the subject.

40. The method of claim 39, wherein the inhibitor of GHR signaling is a protein or an antibody.

41. The method of claim 40, wherein the inhibitor of GHR signaling is Pegvisomant.

42. A method of extending lifespan of a transplant, the method comprising contacting a donor organ obtained from a deceased subject with an inhibitor of insulin-like growth factor 1 receptor (IGF-1R) signaling and/or an inhibitor of growth hormone receptor (GHR) signaling prior to transplant of the donor organ into a subject.

43. The method of claim 42, wherein contacting the donor organ with the inhibitor comprises perfusing the donor organ with a composition comprising the inhibitor of IGF-1R signaling and/or the inhibitor of GHR signaling.

44. The method of claim 42 or claim 43, comprising contacting the donor organ with an inhibitor of IGF-1R signaling.

45. The method of claim 44, wherein the inhibitor of IGF-1R signaling comprises a small molecule inhibitor.

46. The method of claim 45, wherein the inhibitor of IGF-1R signaling comprises a small molecule inhibitor selected from Linsitinib, NVP-AEW541, GSK1904529A, NVP-ADW742, BMS-536924, Ceritinib, AG-1024, GSK1838705A, BMS-754807, PQ 401, Ginsenoside Rg5, Ceritinib dihydrochloride, XL228, Brigatinib, Picropodophyllin, AZD3463, Insulin Degludec, S961, and Nordihydroguaiaretic acid (NGDA).

47. The method of claim 44, wherein the inhibitor of IGF-1R signaling comprises an antibody.

48. The method of claim 47, wherein the antibody is selected from Teprotumumab, Dalotuzumab, Figitumumab, Ganitumab, Robatumumab, and Cixutumumab.

49. The method of claim 42 or claim 43, comprising contacting the donor organ with an inhibitor of GHR signaling.

50. The method of claim 49, wherein the inhibitor of GHR signaling is a protein or an antibody.

51. The method of claim 50, wherein the inhibitor of GHR signaling is Pegvisomant.

52. The method of any one of claims 42-51, wherein the donor organ is a heart, a lung, or a kidney.

53. The method of claim 52, wherein the donor organ is a kidney.

54. A method of extending kidney lifespan in a subject having kidney dysfunction, the method comprising providing an inhibitor of insulin-like growth factor 1 receptor (IGF-1R) signaling and/or an inhibitor of growth hormone receptor (GHR) signaling to a subject having kidney dysfunction.

55. The method of claim 54, wherein the kidney dysfunction is selected from hyperfiltration, glomerular injury, reduced nephron number, and glomerular disease.

56. The method of claim 55, wherein the kidney dysfunction is hyperfiltration, wherein the hyperfiltration is induced by hypertension, partial nephrectomy, kidney transplantation, chronic kidney disease, diabetes, and/or obesity.

57. The method of claim 56, wherein the kidney dysfunction is a glomerular disease, wherein the glomerular disease is selected from focal segmental glomerulosclerosis (FSGS), acute glomerulonephritis, crescentic glomerulonephritis, IgA nephropathy, lupus nephritis, cryoglobulinemia, Membranoproliferative glomerulonephritis (MPGN) transplant glomerulopathy, membranous glomerulopathy, Genetic glomerular disease, and minimal change disease.

58. The method of any one of claims 54-57, comprising providing an inhibitor of IGF-1R signaling to the subject.

59. The method of claim 58, wherein the inhibitor of IGF-1R signaling comprises a small molecule inhibitor.

60. The method of claim 59, wherein the inhibitor of IGF-1R signaling comprises a small molecule inhibitor selected from Linsitinib, NVP-AEW541, GSK1904529A, NVP-ADW742, BMS-536924, Ceritinib, AG-1024, GSK1838705A, BMS-754807, PQ 401, Ginsenoside Rg5, Ceritinib dihydrochloride, XL228, Brigatinib, Picropodophyllin, AZD3463, Insulin Degludec, S961, and Nordihydroguaiaretic acid (NGDA).

61. The method of claim 60, wherein the inhibitor of IGF-1R signaling comprises an antibody.

62. The method of claim 61, wherein the antibody is selected from Teprotumumab, Dalotuzumab, Figitumumab, Ganitumab, Robatumumab, and Cixutumumab.

63. The method of any one of claims 54-57, comprising providing an inhibitor of GHR signaling to the subject.

64. The method of claim 63, wherein the inhibitor of GHR signaling is a protein or an antibody.

65. The method of claim 64, wherein the inhibitor of GHR signaling is Pegvisomant.

66. Use of an inhibitor of insulin-like growth factor 1 receptor (IGF-1R) signaling in a method of extending lifespan of a transplant.

67. The use of claim 66, wherein the transplant is from a deceased donor or a living donor.

68. The use of claim 66 or claim 67, wherein the inhibitor of IGF-1R signaling comprises a small molecule inhibitor.

69. The use of claim 68, wherein the inhibitor of IGF-1R signaling comprises a small molecule inhibitor selected from Linsitinib, NVP-AEW541, GSK1904529A, NVP-ADW742, BMS-536924, Ceritinib, AG-1024, GSK1838705A, BMS-754807, PQ 401, Ginsenoside Rg5, Ceritinib dihydrochloride, XL228, Brigatinib, Picropodophyllin, AZD3463, Insulin Degludec, S961, and Nordihydroguaiaretic acid (NGDA).

70. The use of claim 66 or claim 67, wherein the inhibitor of IGF-1R signaling comprises an antibody.

71. The use of claim 70, wherein the antibody is selected from Teprotumumab, Dalotuzumab, Figitumumab, Ganitumab, Robatumumab, and Cixutumumab

72. Use of an inhibitor of growth hormone receptor (GHR) signaling in a method of extending lifespan of a transplant.

73. The use of claim 72, wherein the transplant is from a deceased donor or a living donor.

74. The use of claim 72 or claim 73, wherein the inhibitor of GHR signaling is a protein or an antibody.

75. The use of claim 74, wherein the inhibitor of GHR signaling is Pegvisomant.
